(19) United States
(12) Patent Application Publication
(10) Pub. No.: US 2015/0153325 A1
(43) Pub. Date: Jun. 4, 2015

(54) TOXIN DETECTION USING STEM CELL DERIVED NEURONS

(71) Applicants: Patrick MCNUTT, (US); Ian GUT, Gunpowder, MD (US); The united States of America as represented by the Secretary of the Army, on Behalf of U.S. Army, Fort Detrick, MD (US)

(72) Inventors: Patrick McNutt, Gunpowder, MD (US); Ian Gut, Gunpowder, MD (US)

(21) Appl. No.: 14/400,487

(22) PCT Filed: May 10, 2013
(86) PCT No.: PCT/US13/40641

§ 371 Date: Nov. 11, 2014

Related U.S. Application Data
(60) Provisional application No. 61/688,735, filed on May 11, 2012.
(60) Provisional application No. 61/646,274, filed on May 12, 2012.

(51) Int. Cl. G01N 33/50

(52) U.S. Cl. CPC G01N 33/5014; G01N 2800/709

(57) ABSTRACT

The present invention relates to neurons derived from a progenitor cell population, capable of forming synapse and producing neurotransmitter signals. Further, methods of producing such neurons and methods of detecting toxin activity using such neurons are disclosed.

### CROSS-REFERENCE TO RELATED APPLICATION

This application claims priority to and the benefit of U.S. Patent Application No. 61/646,274, filed May 12, 2012 and U.S. Patent Application No. 61/688,735, filed May 11, 2012, the disclosures of which are entirely hereby incorporated herein by reference.

### FIELD OF THE INVENTION

The present invention relates to synaptic forming neurons derived from a stem cell population and methods of using the synaptic forming neurons to detect toxins.

### BACKGROUND OF THE INVENTION

Neurotoxins are compounds that adversely affect the nervous system. Typically, neurotoxins act by mechanisms that inhibit neuron processes ranging from membrane depolarization to inter-neuron communication. Neurotoxin exposure can result in nervous system arrest or even nervous tissue death. The onset of symptoms upon neurotoxin intoxication can vary between different toxins, being on the order of hours to years. The speed of recover from neurotoxin intoxication increases with timely medical intervention. Thus, sensitive and rapid toxin detection and diagnostic methods are critical.

Due to the nature of neurotoxins, detection methods need to be sensitive, specific, capable of use with complex samples, detect toxin activity, easy to use, and cost effective. For some neurotoxins, suitable detection methods rely on the use of animals and take weeks to provide results. A commonly used animal based detection method is the mouse bioassay. Despite many attempts to replace the use of animals, the mouse bioassay continues to be relied upon because it is capable of modeling all aspects of neurotoxin intoxication including binding, translocation, and activity. Such animal based detection methods have many drawbacks, including, long assay times, the requirement of special animal facilities, the requirement of specially trained staff, substantial variation in results, and high cost.

Accordingly, a need exists for toxin detection compositions and methods that are sensitive, specific, capable of use with complex samples, capable of detecting toxin activity, easy to use, and cost effective. Further, there is a need for detection methods that do not rely on the death of animals. The present invention provides compositions and methods for toxin, including neurotoxin, detection that are sensitive, specific, capable of use with complex samples, capable of detecting toxin activity, easy to use, cost effective, and do not rely on the death of animals.

### SUMMARY OF THE INVENTION

The present invention is related to compositions and methods of detecting toxins using stem cell derived neurons. Neurons derived from stem cells mimic natural in vivo neurons biologically and physiologically and are useful in detecting femtomolar amounts of toxin. Such neurons may be derived from any totipotent, pluripotent, or multipotent stem cell that can differentiate into a functions neuron capable of forming synapses and releasing neurotransmitters. Suitable stem cells also include embryonic, adult and induced pluripotent stem cells as well as those known in the art or yet to be discovered.

The derived neurons are useful in detecting toxin activity. The present invention includes a method of detecting toxin activity by contacting a sample to a neuron population, and detecting the presence of toxin activity. Suitable neuron populations include neurons capable of synaptic activity, and those derived from stem cells. The activity of the toxin in the neuron population contacted with the sample is compared to the toxin activity in a control neuron population. The control neuron population may be the same neuron population where the toxin activity was detected before and after sample contact. Alternatively, the control neuron population may be a neuron population cultured in parallel, but not contacted with the sample. A difference in the toxin activity of the contacted neuron population as compared to the control neuron population is indicative of a toxin containing sample.

Stem cell derived neurons of the invention are susceptible to toxin levels of about 600, 500, 400, 300, 200, 100, 90, 80, 75, 70, 65, 60, 55, 50, 45, 40, 35, 30, 25, 20, 15, 10, 5, 1, 0.5, 0.1, 0.05, 0.01, 0.005, 0.001 pM or less. Aspects of the invention include stem cell derived neurons that can uptake about 600, 500, 400, 300, 200, 100, 90, 80, 75, 70, 65, 60, 55, 50, 45, 40, 35, 30, 25, 20, 15, 10, 5, 1, 0.5, 0.1, 0.05, 0.01, 0.005, 0.001 pM or less. Some aspects of the invention include stem cell derived neurons that are not susceptible to toxin levels of about 600, 500, 400, 300, 200, 100, 90, 80, 75, 70, 65, 60, 55, 50, 45, 40, 35, 30, 25, 20, 15, 10, 5, 1, 0.5, 0.1, 0.05, 0.01, 0.005, 0.001 pM or less.

Aspects of the invention include detecting toxin levels of about 600, 500, 400, 300, 200, 100, 90, 80, 75, 70, 65, 60, 55, 50, 45, 40, 35, 30, 25, 20, 15, 10, 5, 1, 0.5, 0.1, 0.05, 0.01, 0.005, 0.001 pM or less. Some aspects of the invention include detecting toxin activity at toxin levels of about 600, 500, 400, 300, 200, 100, 90, 80, 75, 70, 65, 60, 55, 50, 45, 40, 35, 30, 25, 20, 15, 10, 5, 1, 0.5, 0.1, 0.05, 0.01, 0.005, 0.001 pM or less of a toxin.

Aspects of the invention include detecting any toxin capable of intoxicating neurons. While any toxin may be detected using the compositions and methods of the invention, exemplary toxins include, without limitation botulinum toxin, tetanus toxin, latrotoxin, shiga toxin, tetrodotoxin, conotoxin, and combinations thereof. Exemplary botulinum toxins include, without limitation, serotype /A, serotype /B, serotype /C, serotype /D, serotype /E, serotype /F, serotype /G, or combinations thereof.

Samples used with the invention include any sample capable of being contacted with a population of neurons. Suitable samples include pure and complex samples. Such samples include, without limitation, purified toxin, a partially purified toxin, unpurified toxin, bulk toxin, formulated toxin, cosmetics toxin formulation, clinical toxin formulation, raw food, cooked food, partially cooked food, processed food, or combinations thereof.

In some aspects, the sample is taken from a subject. Such samples may include, without limitation, tissue, saliva, excretion, feces, blood, urine, or combinations thereof.

Other aspects of the invention include methods of screening for a molecule able to alter toxin activity. Suitable methods include contacting a neuron population with toxin to form an exposed neuron population, detecting the activity of the toxin in the exposed neuron population; contacting the exposed neuron population with a test molecule to form a test neuron population; measuring the toxin activity of the test neuron population; comparing the toxin activity of the test neuron population to the toxin activity of the exposed neuron population; and, correlating a decrease in activity to a molecule capable of inhibiting the toxin activity, or correlating an increase in activity to a molecule capable of enhancing toxin activity. Suitable neuron populations include neurons capable of synaptic activity, and those derived from stem cells. Suitable test molecules include any known in the art or yet to be discovered. Exemplary test molecules include, without limitation, a polypeptide, nucleic acid, synthetic molecule, or combinations thereof.

Aspects of the invention include detecting neurotoxicity. Such neurotoxicity may be detected by measuring the metabolic activity of a derived neuron population that has been contacted with a sample in comparison to a control. A difference in metabolic activity is correlated to neurotoxicity.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows that suspension-adapted ESNs remain mitotically stable and express markers of pluripotency. FIG. 1A shows ESC yields during the first five passages after transition to suspension culture. FIG. 1B shows flow cytometry data demonstrating no substantive change in Oct3/4 expression in three different mESC lines across 25 passages in suspension culture (n=6 for each ESC line). FIG. 1C shows the actual yields (black, filled circles) for a representative R1 ESC culture between 5-30 passages after suspension adaptation. Theoretical yields of ESCs (red squares, filled) and NPCs (red, circles, not filled) over 25 passages are also presented. LIF indicates leukocyte inhibitor factor. Error bars indicate standard deviation.

FIG. 2 shows the timeline from production of suspension-adapted ESCs to neuronal maturation. LIF: leukocyte inhibitory factor; RA: retinoic acid. The presence or absence of RA or LIF is marked by a + or −.

FIG. 3 shows rotary conditions increase NPC yield during differentiation. Bright-field images of ESCs differentiated under static (FIG. 3A) or rotary (FIG. 3B) conditions (45 rpm) are shown. Rotary conditions produced spherical aggregates without agglomeration. FIG. 3C graphically illustrates average NPC yields for each cell line at DIV 0. Rotary culture of cell aggregates improves neural progenitor cell recovery approximately three-fold (p>0.001, n=5).

FIG. 4 shows the immunological characterization of neuron maturations. ESNs were evaluated at indicated days after plating (DIV) for localization of the dendritic marker MAP2 (red), the axonal marker MAP-tau (green) and presynaptic marker synapsin 1 (white).

FIG. 5 shows that ESNs develop large, complex axodendritic arbors by DIV 21. The extent of neuronal network complexity and development was imaged over approximately 9 cm² at DIV 21 by wide-field fluorescent microscopy. Red, dendritic marker MAP2; green axonal marker MAP-tau. The apposition of mature dendritic and axon processes is demonstrated as co-localized staining (yellow).

FIG. 6 shows the characterization of RNA-seq data including quantification of the fifty most abundant transcripts expressed in DIV ESNs (n=5 cultures). Neurotypic (red, solid) and synaptic function (red, checked) genes are indicated. Error bars indicate standard deviations.

FIG. 7 shows depolarization elicits reversible Ca²⁺ influx. Ca²⁺ uptake was evaluated following membrane depolarization by KEB (FIGS. 7A and 7B) or three cycles of electrical field stimulation (10 Hz, 30 sec) (FIGS. 7C and 7D). Time-lapse imaging of Ca²⁺ uptake (FIGS. 7A and 7C) under depolarizing conditions, measured using the fluorescent reporter GCaMP3 (FIG. 7A; genetically encoded) or Fluo-4 (FIG. 7C). Solid black bars in FIG. 7A and FIG. 7C indicate the duration of treatment. Baseline and peak fluorescent intensities are demonstrated by the increase in GCaMP3 (FIG. 7B) or Fluo-4 (FIG. 7D) fluorescence. Error bars indicate standard error (FIG. 7A and FIG. 7C).

FIG. 8 shows that ESNs exposed to glutamate exhibit acute and chronic sequelaea mediated by GluRs. FIG. 8A shows Fluo-4 measurements of Ca²⁺ uptake following the addition of glu (green, solid circles), GABA (black, open circles) or vehicle control (black, open triangles; n≧19 for all samples). K+ (red, solid squares) was used as a positive control. Error bars indicate standard error. FIG. 8B shows representative images of Ca²⁺ uptake in ESNs incubated with Fluo-4 and treated with 200 μM glu. FIG. 8C shows Glu toxicity (200 μM) in ESNs is time-dependent (n=6 for each condition). Data are expressed as fold change relative to control populations. Error bars indicate standard deviation. FIG. 8D shows co-administration of ionotropic GluR antagonists APV and CNQX (A/C) blocks glutamate-induced neuron death at 2 h. * indicates p<0.05 between control and indicated condition. A indicates p<0.05 between glutamate and glutamate plus A/C. (+) and (−) indicate the presence or absence of glutamate or APV/CNQX, respectively. Data are expressed as fold change relative to 0 μM glutamate. FIG. 8E shows neurite degeneration and varicosity formation 24 h after exposure to 200 μM glutamate.

FIG. 9 shows that LTX treatment results in acute Ca²⁺ influx and loss of metabolic activity. FIG. 9A shows intracellular of Ca²⁺ levels correlate to LTX dose at 20 min after exposure (n=8). FIG. 9B shows longitudinal characterization of Ca²⁺ uptake over a 20 min period after LTX addition (n=8). The dotted line in FIGS. 9A and 9B indicates the EC₅₀ (representing the dose of LTX that resulted in 50% of the maximum fluorescence) of 174.9 pM. Error bars indicate the standard error. FIG. 9C shows dose-dependent reduction in ESN viability 30 min after LTX addition (n=8). The data are expressed as fold-change relative to 0 μM glutamate. Error bars indicate standard deviations.

FIG. 10 shows ESNs exhibit sensitive and specific responses to BoNTs. FIG. 10A shows log:lin plot calculated from densitometric measurements of SNARE cleavage after a 24 h exposure to 0-17,800 pM BoNT/B, BoNT/C, BoNT/D, BoNT/F and BoNT/G (n≧5 for all time points). FIG. 10B shows fractional cleavage of SNAP-25 after exposing ESNs to 6.7 or 67 pM BoNT/A under depolarizing (K+) or basal (BEB or NB+B27) conditions (n=5). Error bars indicate standard error.

FIG. 11 shows the Kinetics of SNAP-25 cleavage following internalization of BoNT/A under different conditions. FIG. 11A shows DIV 14-18 ESNs in 6 cm dishes were treated with 0.67-670 pM BoNT/A in B27-NBA medium for 3 h (white columns) or 24 h (gray columns). For the 3 h treatment, cells were washed twice to remove toxin at 3 h and incubated for an additional 21 h. All treatments were harvested at 24 h and the percent of cleaved SNAP25 was determined by densitometry of western blots. FIG. 11B shows SNAP-25 cleavage in embryonic stem cell-derived neurons (ESNs) between 24 to 96 h after a 3 h exposure to 6.7 or 67 pM BoNT/A. FIG. 11C shows an evaluation of the rate of SNAP-25 cleavage, measured as percent-cleaved SNAP-25 per hour, averaged across each time point. For all experiments, n=5 or more replicates. FIG. 11D shows a drug discovery treatment paradigm designed around kinetics of BoNT/A internalization and SNAP-25 cleavage in ESNs. FIG. 11E-G show additional dose and kinetics of SNARE protein cleavage following intoxication with BoNT serotypes. The term 1 tissue culture unit (TCU)=1 U in 50 μL. The use of TCUs allows the adjustment of toxin activity measurements based on the specific activity of a given lot, and easy comparison among lots with different specific activities. FIG. 11E shows log:lin dose response curves to mouse lethal units (U) measured by gel mobility shift assays (immunoblot) 24 h after toxin addition. The bottom axis is in TCUs. FIG. 11F graphically illustrates the kinetics of SNAP-25 cleavage following a 3 hr intoxication with BoNT/A. FIG. 11G shows a schematic of the assay used in FIG. 11F.

FIG. 12 shows LTX but not K⁺ rescues SNAP-25 expression within 48 h in BoNT/A-intoxicated ESNs. BoNT/A-intoxicated ESNs were treated with 60 mM K⁺ or 400 pM LTX for designated times, and SNAP-25 cleavage was evaluated after 48 h by densitometry of western blots. Syntaxin is shown as a loading control.

FIG. 13 shows the kinetics of acute Ca2+ influx mediated by treatment with LTX or K⁺. FIG. 13A shows increased Ca2+ influx (Fluo-4; green) peaked rapidly in response to 60 mM K+ and returned to basal levels within 120 s (upper panels); 6.5 min treatment with 400 pM of LTX resulted in rapid increase of Ca2+ with sustained intensity for over 570 s (lower panels). FIG. 13B shows a quantitative summary of Ca2+ response following addition (down arrows) of 60 mM K+ (closed diamonds; dashed arrows) or 400 pM LTX (open diamonds; solid arrows) from a single field of view. Whereas the K⁺-induced Ca2+ response returned to basal levels prior to washout (up arrows), the LTX-induced Ca2+ response continued to increase through 570 s. Scale bar=10 μm.

FIG. 14 shows a comparison of neuronal morphology changes and total protein yield after treatment with K+ or LTX. DIV 22 ESNs were treated with either 60 mM K+ (top) or 400 pM LTX in BEB (bottom), then perfused for 30 s with BEB. Differential interference contrast images were taken of the same field at the indicated time points (FIG. 14A). Scale bar=10 μm. FIG. 14B shows total protein yield from cultures (n=3) treated with 400 pM LTX for 6.5 or 13 min was decreased by approximately 50% relative to untreated cultures.

FIG. 15 shows varicosity formation in calcein-labeled ESNs over time FIG. 15A. Top panels, low magnification image; bottom panels, high magnification of boxed area in upper panel. Arrows identify representative varicosities that appeared within 24 h and showed a substantive decrease in area by 48 h after treatment. FIG. 14B shows that baricosities appear as soon as 22 min after addition of LTX. All images were of the same field of view for all time points. Scale bar for all images=10 μm.

FIG. 16 shows representative whole cell patch clamp recordings demonstrating elicited electrical responses in DIV 18+ ESNs. Voltage-step protocol demonstrating a fast-activating, fast-inactivating inward Na⁺ current that is inhibited by addition of TTX (FIG. 16A). Vm=−75 mV. Voltage-step protocol demonstrating a delayed rectifier K⁺ current that is inhibited by addition of TEA (FIG. 16B). Vm=−75 mV. Current clamp recordings showing repeated overshooting action potentials are evoked by injection of a 75 pA current (FIG. 16C). Voltage-clamp recordings showing action potential burst and sustained depolarization block following addition of 10 μM glutamate (FIG. 16D). Voltages are adjusted for a liquid junction potential of −15 mV.

FIG. 17 shows the expression of GluRs. DIV 16 ESN lysates were evaluated for the presence of select GluRs by immunoblot (FIG. 17A). SNAP-25 is present as a measure of GluR relative abundance to a highly expressed neuronal protein. ESNs were evaluated for the compartmentalization of GRIN2A/B (green; FIGS. 17B and 17C) in either the dendrites (MAP2, red; FIG. 17B) or axons (TAU protein, red; FIG. 170). The nucleus is indicated by DAPI (blue) staining in the merged images. DIV 18 ESNs were evaluated for the compartmentalization of GRIA1 (FIG. 17D, green) in either the dendrites (FIG. 17D, MAP2, white) or axons (FIG. 17D, TAU protein, red). Markers of significance are per methods section. Results are averaged among three differentiations.

FIG. 18 shows the characterization of functional glutamatergic synaptic activity. FIG. 18A shows representative voltage-clamp recordings from DIV 21 ESNs at resting membrane potential. Robust sEPSCs (top) were eliminated by treatment with TTX (middle), although frequent mESPCs could still be observed. Average sEPSC trace (n=164 events) and mEPSC trace (n=52 events) are presented beside recordings. FIG. 18B shows sample traces from a single neuron showing AMPAR-induced currents at three different holding potentials and I-V plot of evoked AMPAR EPSCs (n=8). Kainate (KA) was used as a low-desensitizing AMPAR agonist. FIG. 18C shows sample traces and I-V plot of evoked NMDAR EPSCs. NMDA-induced currents elicit a voltage-dependent block in the presence of 1 mM Mg²⁺ (right; filled circles; n=12) that becomes linear in Mg²⁺-free medium (open triangles; n=8).

FIG. 19 shows that agonists of GluRs evoke Ca²⁺ uptake. FIG. 19A shows that ESNs treated with 200 μM glutamate underwent a substantial increase in Ca²⁺-mediated Fluo4 fluorescence within 15 s. FIG. 19B shows a glutamate dose-dependent Ca²⁺ uptake. Pretreatment with Gd³⁺ (50 μM) blocked axonal Ca²⁺ uptake. FIG. 19C shows the effect of GluR agonists and neuromodulatory chemicals on Ca²⁺ uptake at 30 s after agonist addition. FIG. 19D shows the quantification of GluR agonists on Ca²⁺ uptake in the presence (+) and absence (−) of APV (A, 50 μM) and CNQX (C, 10 μM) at 30 s after agonist addition. In FIG. 19B-D, data are expressed as the fluorescence change relative to non-stimulated conditions at 30 s after agonist addition. Markers of significance are per methods section. Results are averaged among three differentiations.

FIG. 20 shows that glutamate treatment causes time- and dose-dependent decreases in neuron viability. Glutamate dose-dependent reduction in ESN viability over time during a tonic (FIG. 20A) or following a 5-min (FIG. 20B) glutamate treatment. All data points were significantly different from 0 h (P<0.001). Quantification of glutamate-induced reduction in neuronal viability in the presence of APV (50 μM), CNQX (10 μM) and/or Gd³⁺ (50 μM) (FIG. 20C). Markers of significance are per methods section relative to glutamate alone. D. APV provides a dose-dependent blockade of cell death at 6 h after a 5 min NMDA treatment (FIG. 20D). In FIG. 20A-D, data are expressed as percent viability relative to 0 μM glutamate (FIG. 20A-C) or NDMA (FIG. 20D) and are averaged among 8 independent differentiations. Excitotoxicity evoked by a 5 min exposure to glutamate or NMDA is potentiated in 0 mM Mg²⁺ (p<0.05) and inhibited in 5 mM Mg²⁺ (p<0.001) compared to 1 mM Mg²⁺ (FIG. 20E). Glutamate-induced cell death is blocked by substitution of Ba²⁺ for Ca²⁺ (p<0.01) (FIG. 20F). In FIG. 20E-F, data are the average of ∝12 biological replicates.

FIG. 21 depicts the effects of glutamate treatment on neuron morphology. FIG. 21A shows a time-course evaluation of varicosity formation and neuronal degeneration within ESNs treated with 12.5 μM glutamate. At indicated time points axons and dendrites were visualized with TAU (green) and MAP2 (red), respectively. The top panel is a merge of axon, dendrites, and nuclear labeling (blue) with the bottom panel displaying dendrites alone for clarity. FIG. 21B shows representative SEM micrographs of neurons 6 h after addition of vehicle (left panels) or 200 μM glutamate (right panels).

FIG. 22 shows glutamate activation of cell death pathways. FIG. 22A shows representative photomicrographs of viable, apoptotic or necrotic neurons stained with Hoechst 33342 (blue) and propidium iodide (PI, red). Results are averaged from three independent experiments. FIG. 22B shows the quantification of dose-dependent cell death over time (n 200 nuclei per dose). The planar area of Hoechst 33342 stain was calculated from the average nuclear diameter of apoptotic (3.5 μM glutamate) or necrotic neurons (3000 μM glutamate) (FIG. 22C). Markers of significance are per methods section. In FIG. 22B-C, the results are from >600 neurons per differentiation, averaged over three 3 independent differentiations.

FIG. 23 shows an evaluation of small molecule inhibitors for glutamate-induced neurotoxicity. The addition of APV (A, 50 μM) and CNQX (C, 10 μM) blocks glutamate-induced toxicity at 6 h (FIG. 23A). (̂ indicates P<0.01 between glutamate only and glutamate supplemented with APV/CNQX; (+) and (−) indicate the presence or absence of glutamate or APV/CNQX). FIG. 23B shows dose-dependent blockade of glutamate-induced cell death by APV and CNQX at 6 h. In FIGS. 23A and 23B, the data are expressed as percent viability relative to 0 μM glutamate. Prophylactic and co-administration of an NTF cocktail reduced neuron death at 2 and 6 h after glutamate addition (FIG. 23C). Data are expressed as the fold change in viability of the NGF-pretreated ESNs relative to the glutamate-treated ESNs at the indicated concentration. The dashed line represents the normalized viability of glutamate-treated ESNs at the indicated concentrations. The data are combined from 6 independent differentiations.

FIG. 24 shows that ESNs can be used to detect tetanus toxin. In particular, FIG. 25 shows an immunoblot of ESNs exposed to 150 pM tetanus for 24 h results in nearly complete loss of VAMP2 signal. The loss of signal is due to the rapid proteolytic clearance of the cleaved fragment of VAMP-2.

FIG. 25 shows that LTX treatment results in dose-dependent Ca²⁺ influx. FIG. 25A shows that intracellular Ca²⁺ levels correlate to LTX dose at 20 min after exposure (n=8). FIG. 25B shows longitudinal characterization of Ca²⁺ uptake over a 20 min period after LTX addition (n=8). A, B. Dotted line indicates the EC₅₀.

FIG. 26 graphically depicts Representative data demonstrating ability of small molecules to mitigate LTX-induced Ca²⁺ influx (presumably by sterically blocking ion flux through transmembrane channels).

FIG. 27 shows excitatory post-synaptic currents resulting from action potentials are eliminated by TTX treatment (top). Note that miniature EPSCs are still observed (not visible in this tracing). Treatment with TTX results in complete inhibition of Na⁺ flux through voltage-gated Na⁺ channels under depolarizing conditions (bottom).

FIG. 28 shows whole cell electrophysiological characterization of GABA-A receptor activity. (Top Left). Bicuculine treatment of spontaneously active ESN cultures results in increased rate of action potentials and bursts. (Top right): Addition of muscimol (a GABA-A specific agonist that is the active component of psilocybin) evokes a strong outward Cl− current, consistent with activation of GABA-A receptors. (Bottom right). Addition of GABA silences spontaneous network activity.

### DESCRIPTION OF THE PREFERRED EMBODIMENTS

In accordance with the present invention, compositions and methods using stem cell derived neurons for toxin detection have been discovered. In particular, neurons derived from progenitor cells and grown in suspension cultures respond to femtomolar amounts of toxin. This response is biologically and physiologically similar to that of in vivo neurons. Therefore, the compositions and methods described herein are useful for toxin detection that requires sensitivity, specificity, use with complex samples, detection of toxin activity, ease of use, and cost effectiveness. Furthermore, to compositions and methods described herein reduce the need for animal-based toxicity studies, yet serve to analyze multiple toxin functions.

### I. Compositions

Compositions of the present invention include novel neuron compositions that are susceptible to toxin intoxication to allow assaying of specific toxins. The compositions disclosed herein are useful to conduct methods that can detect femtomolar amounts of toxin in a sample.

The neuron compositions include neurons derived from stem cells. In some aspects neurons are derived from stem cells in a suspended culture. In other aspects neurons are derived from stem cells in adherent cultures, such as on gelatin or in the presence of mouse embryonic fibroblasts. The neurons may be derived from any stem cell capable of differentiating into a neuron. Suitable stem cells may be totipotent cells, pluripotent cells, or multipotent cells. The stem cells may be embryonic stem cells, adult stem cells, induced pluripotent stem cells, or other stem cell known in the art or yet to be discovered.

The stem cell or neuron populations of the invention may be genetically modified. Such genetic modification may include adding, deleting, enhancing, or silencing at least one specific gene or protein or a combination thereof. The genetic modification may be present in the stem cell before derivation or introduced after derivation into neurons. As such, the genetic modification may be introduced stably or transiently. The introduction of genetic modifications using cultured stem cells and derived cell types, such as neurons, is well established in the art.

Aspects of the present invention include stem cell derived neurons and neuron populations thereof that are susceptible to toxin intoxication. In some aspects, the stem cell derived neurons or neuron populations thereof are susceptible to toxin intoxication by about 700 pM or less, about 500 pM or less, about 400 pM or less, about 300 pM or less, about 200 pM or less, or about 100 pM or less of a toxin. In other aspects, the stem cell derived neurons or neuron populations thereof are susceptible to toxin intoxication by about 90 pM or less, about 80 pM or less, about 70 pM or less, about 60 pM or less, about 50 pM or less, about 40 pM or less, about 30 pM or less, about 20 pM or less, or about 10 pM or less of a toxin. In still other aspects, the stem cell derived neurons or neuron populations thereof are susceptible to toxin intoxication by about 9 pM or less, about 8 pM or less, about 7 pM or less, about 6 pM or less, about 5 pM or less, about 4 pM or less, about 3 pM or less, about 2 pM or less, or about 1 pM or less of a toxin. In yet other aspects, the stem cell derived neurons or neuron populations thereof are susceptible to toxin intoxication by about 0.9 pM or less, 0.8 pM or less, 0.7 pM or less, 0.6 pM or less, 0.5 pM or less, 0.4 pM or less, 0.3 pM or less, 0.2 pM or less, or about 0.1 pM or less of a toxin. In other aspects, such neurons are susceptible to toxin intoxication from about 0.01 pM to about 100 pM, about 0.01 pM to about 75 pM, about 0.01 pM to about 50 pM, about 0.01 pM to about 25 pM, about 0.01 pM to about 20 pM, about 0.01 pM to about 15 pM, about 0.01 pM to about 10 pM, about 0.01 pM to about 5 pM, about 0.001 pM to about 100 pM, about 0.001 to about 75 pM, about 0.001 to about 50 pM, about 0.001 to about 25 pM, about 0.001 to about 20 pM, about 0.001 to about 15 pM, about 0.001 to about 10 pM, or about 0.001 to about 5 pM of toxin.

In other embodiments, the stem cell derived neurons or neuron populations thereof are able to uptake a toxin. Such neurons can uptake about 700 pM or less, about 500 pM or less, about 400 pM or less, about 300 pM or less, about 200 pM or less, or about 100 pM or less of a toxin. In other aspects, the stem cell derived neurons or neuron populations thereof can uptake about 90 pM or less, about 80 pM or less, about 70 pM or less, about 60 pM or less, about 50 pM or less, about 40 pM or less, about 30 pM or less, about 20 pM or less, or about 10 pM or less of a toxin. In still other aspects, the stem cell derived neurons or neuron populations thereof can uptake about 9 pM or less, about 8 pM or less, about 7 pM or less, about 6 pM or less, about 5 pM or less, about 4 pM or less, about 3 pM or less, about 2 pM or less, or about 1 pM or less of a toxin. In yet other aspects, the stem cell derived neurons or neuron populations thereof can uptake about 0.9 pM or less, 0.8 pM or less, 0.7 pM or less, 0.6 pM or less, 0.5 pM or less, 0.4 pM or less, 0.3 pM or less, 0.2 pM or less, or about 0.1 pM or less of a toxin. In other aspects, such neurons can uptake toxin from about 0.01 pM to about 100 pM, about 0.01 pM to about 75 pM, about 0.01 pM to about 50 pM, about 0.01 pM to about 25 pM, about 0.01 pM to about 20 pM, about 0.01 pM to about 15 pM, about 0.01 pM to about 10 pM, about 0.01 pM to about 5 pM, about 0.001 pM to about 100 pM, about 0.001 to about 75 pM, about 0.001 to about 50 pM, about 0.001 to about 25 pM, about 0.001 to about 20 pM, about 0.001 to about 15 pM, about 0.001 to about 10 pM, or about 0.001 to about 5 pM of toxin.

In other embodiments, the stem cell derived neurons or neuron populations thereof are not susceptible to a toxin. Such neurons are not susceptible to a toxin at about 700 pM or less, about 500 pM or less, about 400 pM or less, about 300 pM or less, about 200 pM or less, or about 100 pM or less of a toxin. In other aspects, the stem cell derived neurons or neuron populations thereof are not susceptible to a toxin at about 90 pM or less, about 80 pM or less, about 70 pM or less, about 60 pM or less, about 50 pM or less, about 40 pM or less, about 30 pM or less, about 20 pM or less, or about 10 pM or less of a toxin. In still other aspects, the stem cell derived neurons or neuron populations thereof are not susceptible to a toxin at about 9 pM or less, about 8 pM or less, about 7 pM or less, about 6 pM or less, about 5 pM or less, about 4 pM or less, about 3 pM or less, about 2 pM or less, or about 1 pM or less of a toxin. In yet other aspects, the stem cell derived neurons or neuron populations thereof are not susceptible to a toxin at about 0.9 pM or less, 0.8 pM or less, 0.7 pM or less, 0.6 pM or less, 0.5 pM or less, 0.4 pM or less, 0.3 pM or less, 0.2 pM or less, or about 0.1 pM or less of a toxin. In other aspects, such neurons are not susceptible to a toxin from about 0.01 pM to about 100 pM, about 0.01 pM to about 75 pM, about 0.01 pM to about 50 pM, about 0.01 pM to about 25 pM, about 0.01 pM to about 20 pM, about 0.01 pM to about 15 pM, about 0.01 pM to about 10 pM, about 0.01 pM to about 5 pM, about 0.001 pM to about 100 pM, about 0.001 to about 75 pM, about 0.001 to about 50 pM, about 0.001 to about 25 pM, about 0.001 to about 20 pM, about 0.001 to about 15 pM, about 0.001 to about 10 pM, or about 0.001 to about 5 pM of toxin.

The present invention contemplates any toxin capable of intoxicating the stem cell derived neurons of the invention. Such toxins may include, without limitation, indirect as well as direct toxins. Indirect toxins are those toxins that alter neurotransmitter release or uptake, such as causing a toxic level of Ca2⁺. Direct toxins include those toxins that directly act on a neuron, such as by uptake into a neuron or attaching to neuron membranes. While the invention encompasses any toxin capable of altering neuron homeostasis or normal neuron function, examples of toxins include, without limitation, Ablomin, Aconitine, Aconitum, Aconitum anthora, AETX, Agitoxin, Aldrin, Alpha-neurotoxin, Altitoxin, Anatoxin-a, Anisatin, Anthopleurin, Apamin, 2-Ethoxyethyl Acetate, Acibenzolar-S-methyl, Acrylamide, Aldicarb, Allethrin, Aluminum (cl or lactate), Amino-nicotinamide(6-), Aminopterin, Amphetamine(d-), Arsenic, Aspartame, Azacytidine(5-), Babycurus toxin 1, Batrachotoxin, Bestoxin, Birtoxin, BmKAEP, BmTx3, Botulinum toxin, Brevetoxin, Para-Bromoamphetamine, Bukatoxin, Benomyl, Benzene, Bioallethrin, Bis(tri-n-butyltin)oxide, Bisphenol A, Bromodeoxyuridine(5-), Butylated Hydroxy Anisol, Butylated hydroxytoluene, Calcicludine, Calciseptine, Carbon disulfide, Charybdotoxin, Para-Chloroamphetamine, Cicutoxin, Ciguatoxin, Clostridium botulinum, Conantokins, Conhydrine, Coniine, Conotoxin, Contryphan, Curare, Cyanide poisoning, Cylindrospermopsin, Cypermethrin, Cadmium, Caffeine, Carbamazepine, Carbaryl, Carbon monoxide, Chlordecone, Chlordiazepoxide Chlorine dioxide, Chlorpromazine, Chlorpyrifos Cocaine, Colcemid, Colchicine, Cypermethrin, Cytosine Arabinoside, Delta atracotoxin, Dendrotoxin, Dexamethasone, Diamorphine hydrochloride, Dieldrin, 5,7-Di hydroxytryptamine, Diisopropyl fluorophosphate, Dimethylmercury, Discrepin, Domoic acid, Dortoxin, DSP-4, Diazepam, DEET, Deltamethrin, Diazinon, Dieldrin, Diethylstilbestrol, Diphenylhydantoin, Epidermal Growth Factor, Ethanol, Ethylene thiourea, Falcarinol, Fluorouracil(5-), Fluazinam, Fluoride, Gabaculine, Ginkgotoxin, Grammotoxin, Grayanotoxin, Griseofulvin, Hainantoxin, Hefutoxin, Helothermine, Heteroscodratoxin-1, Histrionicotoxin, Hongotoxin, Huwentoxin, Haloperiodol, Halothane, Heptachlor, Hexachlorobenzene, Hexachlorophene, Hydroxyurea, Para-Iodoamphetamine, Ibotenic acid, Ikitoxin 5-Iodowillardiine, Imminodiproprionitrile (IDPN), Jingzhaotoxin, Ketamine, Kurtoxin, Latrotoxin, Alpha-Latrotoxin, Lq2, Lead, LSD, Lindane, Maitotoxin, Margatoxin, Maurotoxin, Methanol, Methiocarb, Beta-Methylamino-L-alanine, N-Methylconiine, MPP+, MPTP, Myristicin, Maneb, Medroxyprogesterone, Mepivacaine, Methadone, Methanol, Methimazole, Methylparathion, Monosodium Glutamate, MPTP, Naloxone, Nemertelline, Neosaxitoxin, Nicotine, Naltrexone, 2-Methoxyethanol, Methylazoxymethanol, Methylmercury, Oxidopamine, Ozone, Oenanthotoxin, Oxalyldiaminopropionic acid, Palytoxin, Penitrem A, Phaiodotoxin, Phenol, Phoneutria nigriventer toxin-3, Phrixotoxin, Polyacrylamide, Poneratoxin, Psalmotoxin, Pumiliotoxin, Paraquat, Parathion (ethyl), PBDEs, PCBs (generic), Penicillamine, Permethrin, Phenylacetate Phenylalanine (d,l), di-(2-ethylhexyl)Phthalate, Propylthiouracil, Raventoxin, Resiniferatoxin, Retinoids/vit.A/isotretinoin, Samandarin, Saxitoxin, Scyllatoxin, Sea anemone neurotoxin, Shiga toxin, Slotoxin, SNX-482, Stichodactyla toxin, Salicylate, Taicatoxin, Taipoxin, Tamapin, Tertiapin, Tetanospasmin, Tetanus toxin, Tetraethylammonium, Tetrodotoxin, Tityustoxin, Tricresyl phosphate, Tebuconazole, Tellurium (salts), Terbutaline, Thalidomide, THC, Toluene, Triamcinolone, Tributyltin chloride, Trichlorfon, Trichloroethylene, Triethyllead, Triethyltin, Trimethyltin, Trypan blue, Urethane, Valproate, Vanillotoxin, Veratridine, Vincristine. Preferred toxins included Botulinum toxin, Tetanus toxin, Latrotoxin, Shiga toxin, Tetrodotoxin, Conotoxin, and combinations thereof. Preferred Botulinum toxins include serotype /A, serotype /B, serotype /C, serotype /D, serotype /E, serotype /F, serotype /G, or combinations thereof.

### II. Methods

The present invention provides novel assays for detecting the presence or absence of an active neurotoxin. The methods disclosed herein reduce the need for animal-based toxicity studies. The methods disclosed herein may be used to analyze crude and bulk samples as well as highly purified toxins and formulated toxin products. As non-limiting examples, methods of the invention may be useful for detecting the presence or activity of a toxin in a food or beverage sample; to assay a sample from a human or animal, for example, exposed to a toxin or having one or more symptoms of toxin exposure; to follow activity during production and purification of toxin; to assay formulated toxin products such as pharmaceuticals or cosmetics; or for other reasons that become apparent to a skilled artisan.

The present invention includes methods of detecting toxin activity by contacting a sample to a population of neurons that were derived from progenitor cells and are capable of toxin intoxication. Further, the presence of toxin activity of the contacted neuron population is detected relative to a control neuron population. A difference in the toxin activity of the contacted neuron population compared to the control neuron population is indicative of toxin activity.

Another aspect includes methods of detecting toxin activity by contacting a sample to a neuron population that transiently contains a genetic modification, was derived from progenitor calls, and is capable of toxin intoxication. Further, the presence of toxin activity of the contacted neuron population is detected relative to a control neuron population. A difference in the toxin activity of the contacted neuron population compared to the control neuron population is indicative of toxin activity.

Another aspect includes methods of detecting toxin activity by contacting a sample to a neuron population that stably contains a genetic modification, was derived from progenitor calls, and is capable of toxin intoxication. Further, the presence of toxin activity of the contacted neuron population is detected relative to a control neuron population. A difference in the toxin activity of the contacted neuron population compared to the control neuron population is indicative of toxin activity.

Aspects of the invention include detecting neurotoxicity. Such neurotoxicity may be detected by measuring the metabolic activity of a derived neuron population that has been contacted with a sample in comparison to a control. A difference in metabolic activity is correlated to neurotoxicity. Methods of detecting and measuring metabolic activity are known in the art and contemplated herein.

In other embodiments, the present invention includes methods of screening for a molecule able to modulate toxin activity or intoxication. Such methods include contacting a neuron population with a toxin of interest and detecting the toxin activity. A neuron population exposed to a toxin of interest is contacted with at least one test molecule forming a test neuron population. Toxin activity of the test neuron population is measured relative to a control neuron population that was not contacted with the test molecule. A difference in the toxin activity of the test neuron population compared to the control neuron population is indicative of a molecule capable of modulating activity of the toxin of interest.

The molecule to be tested in the screening method may be a “small” organic compound or may be a macromolecule. The molecule may be of synthetic or biological origin. Exemplary molecules include, without limitation, a polypeptide, such as a growth factor, a neurotoxin, a modified neurotoxin, an antibody or an antibody derivative; a nucleic acid, such as a nucleic acid aptomer; and a polysaccharide, such as a ganglioside or a lectin. In one embodiment, the molecule is a synthetic molecule designed based on the tertiary structure and three dimensional conformation of a peptide or antibody that inhibits a specific toxin uptake into a neuron.

Toxin activity may be detected using any method known in the art. A wide variety of assays may be used to determine the presence of toxin activity, including direct and indirect assays for toxin uptake. Assays that determine toxin binding or uptake properties may be used to assess toxin activity. Such assays include, without limitation, cross-linking assays using labeled toxin. Other non-limiting assays include immunocytochemical assays that detect toxin binding using labeled or unlabeled antibodies; and, immunoprecipitation assays. Antibodies useful for these assays include antibodies selected against the specific toxin in question (e.g. BoNT/A, BoNT/B, BoNT/C, BoNT/D, BoNT/E, BoNT/F, LTX, etc.); antibodies selected against a receptor the toxin binds to, such as FGFR3 or SV2; antibodies selected against a ganglioside, such as GD1a, GD1b, GD3, GQ1b, or GT1b; or antibodies selected against a test molecule. If the antibody is labeled, the binding of the molecule may be detected by various means, including Western blotting, direct microscopic observation of the cellular location of the antibody, measurement of cell or substrate-bound antibody following a was step, or electrophoresis, employing techniques well-known to those of skill in the art. If the antibody is unlabeled, one may employ a labeled secondary antibody for indirect detection of the bound molecule, and detection can proceed as for a labeled antibody.

Assays that monitor the release of a molecule after exposure to a toxin may also be used to assess for the presence of toxin activity. Exemplary assays that may be used include, without limitation, the insulin release assay, measuring inhibition of radio-labeled catecholamine release from neurons, syntaxin cleavage, SNAP-25 cleavage, and others known in the art.

It is envisioned that a wide variety of processing formats may be used in conjunction with the methods disclosed herein, including, without limitation, manual processing, partial automated-processing, semi-automated-processing, full automated-processing, high throughput processing, high content processing, and the like or any combination thereof.

### III. Kits

The present invention provides articles of manufacture and kits containing materials useful for detecting toxins as described herein. The article of manufacture may include a container of a composition as described herein with a label. Suitable containers include, for example, bottles, vials, and test tubes. The containers may be formed from a variety of materials such as glass or plastic. The container holds a composition which is useful for detecting toxins, for example, in samples suspected of containing toxins. The composition includes stem cell derived neurons capable of intoxication by toxins and may further include culture media for maintaining such neurons. The label on the container may indicate that the composition is useful for detecting toxins and may also indicate directions for detection.

### DEFINITIONS

Unless defined otherwise, all technical and scientific terms used herein have the same meaning as is commonly understood by one of ordinary skill in the art. All patents, applications, published applications and other publications are incorporated by reference in their entirety. In the event that there is a plurality of definitions for a term herein, those in this section prevail unless stated otherwise.

As used herein, the term “about” when qualifying a value of a stated item, number, percentage, or term refers to a range of plus or minus ten percent of the value of the stated item, percentage, parameter, or term.

The term “stem cell derived” refers to a population of cells that are the result of induced stem cell differentiation. For example, stem cell derived neurons are a population of neurons resulting from exogenously providing differentiation factors to a stem cell or population of stem cells to promote differentiation into neurons.

As used herein, the term “sample” refers any composition that contains or potentially contains a toxin. A variety of samples may be used with the methods disclosed herein including, without limitation, purified, partially purified, or unpurified toxin; toxin with naturally or non-naturally occurring sequence; recombinant toxin; chimeric toxin containing structural elements from multiple toxin species or subtypes; bulk toxin; formulated toxin product; foods; cells or crude, fractionated or partially purified cell lystates, for example, engineered to include a recombinant nucleic acid encoding a toxin or gene of interest; bacterial, baculoviral and yeast lysates; raw, cooked, partially cooked or processed foods; beverages; animal feed; soil samples; water samples; pond sediments; lotions; cosmetics; and clinical formulations. The term “sample” also encompasses tissue samples, including, without limitation, mammalian tissue samples, livestock tissue samples such as sheep, cow and pig tissue samples; primate tissue samples; and human tissue samples. Such samples encompass, without limitation, intestinal samples, saliva, excretions, feces, urine, blood, samples from wounds, and mucous. It is also contemplated that the term “sample” also encompasses those of a specific environment. Such samples may include soil, water, biomass, plant, tree, air, gas, or combinations thereof.

As used herein, the term “toxin” refers to any substance, molecule, or composition that is capable of intoxicating neurons. Such toxins may include, without limitation, indirect as well as direct toxins. Indirect toxins are those toxins that alter neurotransmitter release or uptake, such as causing a toxic level of Ca2⁺. Direct toxins include those toxins that directly act on a neuron, such as by uptake into a neuron or attaching to neuron membranes.

### EXAMPLES

The following examples are simply intended to further illustrate and explain the present invention. The invention, therefore, should not be limited to any of the details in these examples.

### Example 1

### Materials and Methods

### Reagents

R1, D3 and C57BL/6 ESC lines were obtained from American Type Culture Collection (ATCC, Manassas, Va., USA). Latrotoxin (Sigma-Aldrich, St. Louis, Mo., USA) was resuspended to 300 nM in water and stored at −20° C. Fluo-4 (Invitrogen, Carlsbad, Calif., USA) and Calcein/AM (Invitrogen) were prepared per the manufacturer's instructions. Pure botulinum holotoxin serotypes /A (2.5×108 LD50/mg), /B (1.1×108 LD50/mg), /C (3.5×107 LD50/mg), /D (0.9×108 LD50/mg), IF (0.2×108 LD50/mg) and /G (1.2×107 LD50/mg) were obtained from Meta-biologics (Madison, Wis.) at 1 mg/mL in Ca²⁺/Mg²⁺-free phosphate buffered saline, pH 7.4 (PBS), and stored at −30° C. In the case of BoNT/G, toxin was first activated by 60 min incubation at 37° C. in 0.05 M sodium phosphate buffer (pH 6.5), 0.3 mg/mL TPCK-treated trypsin (Sigma-Aldrich) and 10% glycerol. Activated toxin was diluted 1:1 with soybean trypsin inhibitor and stored at −30° C. until use. α-Latrotoxin (LTX; Sigma-Aldrich) was resuspended to 300 nM in water and stored at −20° C. Mono-sodium glutamate (Sigma-Aldrich) and γ-aminobutyric acid (GABA; Sigma-Aldrich) were resuspended to 20 mM in PBS and stored at 4° C. Solutions were diluted to the indicated concentrations in basal electrophysiologic buffer (BEB; 10 mM glucose, 1 mM MgCl₂, 10 mM HEPES, 2 mM CaCl₂, 3 mM KCl, 136 mM NaCl and 0.1% BSA, pH 7.4, 310±10 mOsm; Sigma-Aldrich). R-2-amino-5-phosphonopentanoate (AVP, 50 μM, Sigma) and 6-cyano-7-nitroquinoxaline-2,3-dione (CNQX, 10 μM, Sigma) were prepared in BEB and added 1 h prior to and concurrent with the addition of glutamate. Fluo-4 (Life Technologies, Carlsbad, Calif.) was prepared per the manufacturer's instructions. Neurons were maintained in BEB during time-lapse imaging. High potassium electrophysiologic buffer (KEB) was prepared similarly to BEB, except with substitutions of 60 mM KCl and 79 mM NaCl. Electrical field stimulation (1 msec, 100 mV pulses at 10 Hz) of neurons on 18-mm cover slips was applied via a field stimulation perfusion chamber (PC-49FS; Warner Instruments, Hamden, Conn.) with a Grass S88 stimulator (Grass Medical Instruments, Quincy, Mass.). Mono-sodium glutamate, 2-amino-3-(5-methyl-3-oxo-1,2-oxazol-4-yl)propanoic acid (AMPA) kainic acid (KA), γ-aminobutyric acid (GABA), N-methyl-D-aspartate (NMDA), gadolinium chloride, and saponin were purchased from Sigma-Aldrich (St. Louis, Mo.). Solutions were diluted to the indicated concentrations in the described buffer at the time of the experiment. Fluo-4, Hoechst 33342, propidium iodide (PI) and PrestoBlue were purchased from Life Technologies (Carlsbad, Calif.) and prepared per the manufacturer's instructions.

### Embryonic Stem Cell Culture and Neuronal Differentiation

Murine embryonic stem cells were maintained and differentiated into neurons (ESNs). ES cells were cultured in suspension in DMEM with 0.1 mM (3-mercaptoethanol, 15% fetal calf serum, nonessential amino acids, L-glutamine and 1000 units/mL LIF. Cell aggregates were dissociated with TrypLE every 48 hours (h) and subpassaged to 1.5×10⁵ cells/mL. Neurons were derived between passages 5 and 30 with differentiating aggregates being maintained in low attachment petri dishes on a rotary shaker at 45 rotations per minute (min.). Cell aggregates were dissociated with TrypLE and plated in N2 medium (Neurobasal-A medium with L-glutamine and N-2 supplement) at 1.5×10⁵ cells/cm² in PDL-coated tissue culture dishes. Cells were washed with N2 medium at 2 and 24 h and subsequently cultured in B27 medium (Neurobasal-A medium with L-glutamine and B27 supplement) starting at DIV 2. For transgenesis, ES cells were transfected with a construct expressing mVenus under control of the CAG promoter using Lipofectamine. Clonal aggregates were expanded and differentiated after 10 days of selection with 250 μg/mL geneticin. In some embodiments, ESNs were plated in PDL-coated 60 mm dishes at 125,000 cells/cm2 or PDL- and laminin-coated 18 mm coverslips at 100,000 cells/cm2 and maintained in Neurobasal-A medium (NBA) with B27 vitamins (Invitrogen, Carlsbad, Calif., USA).

Suspension Adaptation and Continuous mESC Culture

R1, D3 and C57BL/6 cell lines that had been previously maintained in adherent culture with mouse embryonic fibroblasts (MEFs) were thawed and maintained at 37° C. at 5% CO₂ in 90% relative humidity in 10-cm bacterial plates in 10 mL ESM (Knockout DMEM supplemented with 100 μM β-mercaptoethanol, 15% ES qualified fetal calf serum (ATCC), 0.1 mM nonessential amino acids, 2.0 mM L-glutamine and 5000 units/mL penicillin/streptomycin (Life Technologies) and 1000 units/mL recombinant mouse leukemia inhibitory factor (LIF; Chemicon International, Temecula, Calif.)). Alternatively, ESCs were maintained in commercially prepared complete ESC medium (Millipore, Billerica, Mass.). Cells were observed daily and passaged once aggregates became clearly visible (typically 4-8 days). Cells that were adherent or failed to divide during adaptation were discarded. Surviving aggregates were trypsinized, and 1.5×10⁶ cells were inoculated into a fresh 10-cm bacterial dish with 10 mL ESM. ESCs were subcultured every 48 h. For passaging, aggregates were allowed to settle by gravity, washed once with 0.5 mL PBS and dissociated for 3 min at 37° C. with 0.5 ml of TrypLE Express (Life Technologies). Dissociation was terminated with 0.5 mL of ESM, cells were gently triturated and 1.5×10⁶ mESCs were transferred to a fresh 10-cm dish.

### Generation of GCaMP3-Expressing ESCs

Suspension-adapted R1 ESCs were stably transfected with a genetically encoded Ca²⁺ construct (GCaMP3) driven by the synthetic CAG promoter (Addgene plasmid 22692, Cambridge, Mass.). Lipofectamine 2000 at 5 μL, 5 μg plasmid and 5 μL PLUS reagent were prepared per manufacturer's instructions (Invitrogen) in a total volume of 100 μL DMEM and added to 100 μL of 1×10⁶ ESCs/mL in DMEM for 10 min at 37° C. Suspensions were transferred to 10 mL ESM in a bacterial dish and returned to an incubator. Media was changed at 1 day (d) and G418 selection (250 μg/mL; Sigma-Aldrich) started at 2 d. Transient transfection rates exceeded 40%, as estimated from basal fluorescence levels at 2 d. Media was subsequently changed every 4 d until G418-resistant ESC aggregates developed. Stably transfected ES cell aggregates (20-50 per transfected population) were isolated and cultured as above until differentiation.

### Neuronal Differentiation

Following routine sub-passaging, 3.5×10⁶ dissociated ESCs were transferred to 25 mL of differentiation medium (ESM modified with 10% ES qualified fetal calf serum and without LIF) in a 10-cm ultra-low attachment culture dish (Corning, Lowell, Mass.). Differentiating aggregates were maintained on a rotary shaker at 45 rpm at 37° C., 5% CO₂ and 90% relative humidity. Complete media changes were conducted at 48 h intervals, with the addition of 6 μM retinoic acid (Sigma-Aldrich) at 4 and 6 days after starting differentiation.

On DIV 0, aggregates were dissociated with TrypLE Express for 5 min at 37° C. Trypsinization was halted by adding 5 mL of 1% soybean trypsin inhibitor (Life Technologies), the aggregates were gently dissociated by triturating with a 10 mL pipet, and the cell suspension was filtered through a 40-μm cell strainer (Thermo Scientific). Cells were pelleted for 5 min at 300×g, washed in N2 medium (Neurobasal-A medium with 1×N2 vitamins, 2 mM glutamine and antibiotics (Life Technologies)) and counted. Cells were plated at 125,000 (coverslips) or 150,000 cells/cm² (dishes) in N2 medium. Complete washes were conducted at 4 h and 24 h to remove residual serum, gliotrophic factors secreted by glial cells and non-adherent cells, and at 48 h after plating (DIV 2), N2 was replaced with B27 medium (Neurobasal-A supplemented with antibiotics, 2 mM glutamine and 1×B27 vitamins (Life Technologies)). Subsequently, cells underwent full medium changes with B27 on DIV 4 and 8, and then 50% media changes with B27 every fifth day. Glial cell elimination by serum-starvation starting at DIV 0 resulted in the loss of roughly 30% of plated cells between DIV 2-4.

Tissue culture treated dishes ranging in size from 10 cm to 24-well plates were prepared by coating with 0.5 μg/mL poly-D-lysine (PDL, Sigma-Aldrich) for at least 3 h, followed by two quick washes with sterile ddH₂O and storage in N2 at 37° C. until plating.

Coverslips (18 mm, Thermo Fisher Scientific, Waltham, Mass.) were coated with 200-300 μL of 0.5 μg/mL PDL for 24 h at 37° C. followed by 5 μg/mL laminin (Sigma-Aldrich) in Knockout DMEM for 3 h and transferred coated-side up to 12-well dishes. All characterization of differentiated neurons was conducted exclusively with neurons derived from R1 ESCs between 5-30 passages.

### Time-Lapse Confocal Microscopy

Images were collected on a Zeiss LSM-700 confocal microscope with constant-temperature environmental chamber. For Fluo-4 staining, ESNs on 18-mm coverslips were loaded with 1 μM Fluo-4 for 20 min and washed thoroughly. Coverslips were mounted in a Warner (Hamden, Conn.) closed-bath imaging chamber, maintained at 37° C. with a heated stage and perfused with phenol-free Hibernate (Brainbits, Springfield, Ill.). For calcein green staining, cells were incubated with 1 μM calcein green in NBA for 30 min, then washed thoroughly and mounted as above. In both cases, coverslips were imaged at 63× using the 488 laser and manufacturer recommended filter sets. Zen 2009 (Carl Zeiss, Inc, Oberkochen, Germany) was used to determine the mean fluorescence intensity over a fixed area using Fluo-4 and GCaMP3 as indicators for the presence of cytosolic Ca²⁺. Ca²⁺ uptake was induced with the application of KEB or LTX at indicated quantities or by field stimulation. To elicit action potentials by field stimulation, electrical current was applied through platinum wires located in the microscope field of view, using 1 msec, 100 mV pulses. Neurons were stimulated with 3 cycles of approximately 300 action potentials at 10 Hz, with a 10 sec rest. The data were normalized to ΔF/F₀ via the following equation: y=(F_{Δ min}−F_{0 min})/F_{0 min}.

### Immunoblotting

ESN cultures were lysed with 250 μL denaturing cell extraction buffer (Life Technologies) and clarified by centrifugation through a Qiashredder (Qiagen, Valencia, Calif.); total protein concentration was determined by bicinchoninic acid (BCA) analysis (Thermo Scientific, Rockford, Ill.). Fifteen micrograms of total protein was separated on a 10% (BoNT/B, BoNT/D, BoNT/F or BoNT/G) or 12% (BoNT/A or /C) Nupage gel (Life Technologies) with MOPS running buffer. Gels were transferred to PVDF and probed for SNARE proteins with a mouse anti-SNAP-25 antibody (SMI81; Covance, Gaithersburg, Md.), a mouse anti-VAMP2 antibody (Synaptic Systems, Gottingen, Germany), and a mouse anti-Syntaxin-1 (Synaptic Systems) diluted 1:1000 in TBS Superblock with 0.05% Tween-20 (TBST; Life Technologies). Proteins were visualized with goat anti-mouse Alexa-488 labeled antibodies diluted 1:2500 in TBST and imaged with a Versadoc MP4000 (Bio-Rad, Hercules, Calif.).

In another embodiment, ESN cultures were lysed with denaturing cell extraction buffer (Life Technologies) and harvested by scraping. Lysates were vortexed briefly, stored at 4° C. for 15 min, and clarified by centrifugation for 3.5 min at 16,000×g. Total protein concentration was determined by bicinchoninic acid analysis (Thermo Scientific, Rockford, Ill.), and 25 μg of total protein were separated on a 4-12% Nupage gel (Life Technologies). Gels were transferred to PVDF and probed with primary antibodies against GluN 1, GluN2B, GluN1A/B, GluA1-4 and GluR6 (Synaptic Systems Goettingen, Germany) diluted 1:1000 in TBS with 0.05% Tween-20 (TBST; Life Technologies). Proteins were visualized with goat anti-mouse or goat anti-rabbit Alexa-488 labeled antibodies diluted 1:2500 in TBST and imaged with a Versadoc MP4000 (Biorad, Hercules Calif.).

### Plate Reader Based Quantification of Ca²⁺ Uptake

ESNs were plated in either 24- or 48-well dishes, stained, mounted and maintained at 25° C. [25]. Ca²⁺ uptake was induced with the application of chemical stimulation at indicated concentrations. Changes in fluorescence were monitored with a Synergy MX plate reader (BioTek Instruments, Inc., Winooski, Vt.) with excitation of 490/10 nm and emission of 520/10 nm. The data were normalized via the following equation: y=(Fₓ−F₀)/F₀.

### Immunocytochemistry

Coverslips were fixed with 4% paraformaldehyde for 15 min at room temperature, blocked, and permeabilized for 10 min in PBS with 0.1% saponin and 3% bovine serum albumin (BSA) (PBSS). Coverslips were incubated for 1 h with anti-MAP-Tau and anti-MAP2 primary antibodies (Synaptic Systems, Gottingen, Germany) diluted 1:1000 and 1:500, respectively, in PBSS, washed three times with PBSS and incubated for 1 h with goat anti-mouse or anti-rabbit Alexa-labeled secondary antibodies (Invitrogen) diluted 1:500 in PBSS. Coverslips were washed three times in PBSS and incubated for 1 h with antisynapsin 1-Oyster 650 primary antibody (Synaptic Systems) diluted 1:500 in PBSS. Coverslips were washed three times in PBS and mounted with Prolong Gold DAPI mounting media (Life Technologies). Images were collected with a Zeiss LSM 700 confocal microscope or Zeiss LSM 510 confocal microscope and analyzed with Zen 2009 or 2008, respectively.

### Differential Interference Contrast (DIC)/Bright Field Microscopy

For evaluation of neuronal morphology, images were collected on a Zeiss LSM 700 confocal microscope with a constant temperature chamber and analyzed with AxioVision LE release 4.8.1. For evaluation of aggregate morphology, images were collected on a Zeiss AxioOb-server.A1 epi-fluorescence microscope and analyzed with AxioVision release 4.7.1.

### Flow Cytometry

mESCs were collected during routine subculture and washed twice with PBS. Cells were fixed with paraformaldehyde, permeabilized and stained for Oct3/4 expression or with appropriate isotype controls using the Human and Mouse Pluripotent Stem Cell Analysis Kit (BD Biosciences, San Jose, Calif.). Stained cells were analyzed on a BD FACSAria II flow cytometer using manufacturer-specified laser excitation wavelength and emission filter sets. At least 10,000 gated events were recorded and analyzed for each sample. Density plots and fluorescence intensity histograms were generated using FCS Express, and experimental samples were normalizing to isotype controls.

### Quantitation of ESN Viability

ESNs were plated in either 24- or 48-well dishes and maintained under indicated conditions. PrestoBlue (Invitrogen) was added per manufacturer's protocols in fresh NBA-B27, and ESNs were incubated for 45 min at 37° C. Metabolic conversion of PrestoBlue was measured using an excitation of 535 nm and emission of 595 nm with a Synergy MX plate reader.

### Expression Profiling

RNA was harvested from 10-cm dishes of DIV 14 ESNs (n=5) using RNeasy mini kit (Qiagen) and submitted to Expression Analysis (Durham, N.C.) for RNA sequencing (2×25 paired-end, on an Illumina HiSeq 2000 [San Diego, Calif.]). Transcriptome data were aligned using UCSC's mouse knowngene transcriptome with Tophat. Unaligned reads were then aligned to the genome using BWA and merged with the Tophat alignments. Transcript abundances were determined using RSEM v1.1.13 and normalized by calculating the fragments of exon per million mapped reads (FPKM). Normalized data were analyzed for enrichment of canonical pathways, gene function and tissue expression by Ingenuity Pathway Analysis (Ingenuity Systems, Redwood City, Calif.). The most abundant 3000 transcripts were also analyzed for gene functional classification and tissue enrichment using Database for Annotation, Visualization, and Integrated Discovery (DAVID).

### Electrophysiology

DIV 1 8-24 neuron cultures were visualized on an Olympus IX51 microscope (Shinjuku, Tokyo, Japan) equipped with a 40× lens with differential interference contrast optics 5-7 MO pipettes were pulled from capillary glass (Sutter Instruments Novato, Calif.), backfilled with intracellular recording buffer and dipped in Sigmacote® (Sigma) just prior to use. Electrophysiology data was acquired at 20-22° C. with an EPC10 (Heka, Lambrecht/Pfalz, Germany) and Heka Patchmaster 2.53 software. Data analysis and graphing was performed in Heka Fitmaster 2.53, Igor Pro v6 (Wavemetrics Portland, Oreg.) and Prism v6 (Graphpad Software, La Jolla, Calif.). The 10-90% rise time, peak amplitude, and decay kinetics of mEPSCs were calculated with Mini Analysis (Synaptosoft, Inc, Decatur, Ga.).

For characterization of intrinsic voltage-gated responses sEPSCs and current clamp recordings pipettes were filled with an intracellular recording buffer containing (in mM): 140 K-gluconate, 5 NaCl, 2 Mg-ATP, 0.5 Li-GTP, 0.1 CaCl₂, 1 MgCl₂, 1 ethylene glycol-bis(b-aminoethyl ether)-N,N,N,N-tetraacetic acid (EGTA) and 10 HEPES Cultures were bathed in an extracellular recording buffer (ERB) containing (in mM): 140 NaCl, 3.5 KCl, 1.25 NaH₂PO₄, 2 CaCl₂, 1 MgCl₂, 10 Glucose, and 10 HEPES. All buffers were adjusted to pH of 7.3 with NaOH and an osmolarity of 315±10 mOsm with glucose prior to recording.

To characterize current:voltage (I-V) relationships and mEPSC kinetics neurons were bathed in ERB supplemented with 5 μM TTX and 1 μM glycine and patched with electrodes containing (in mM): 125 CsCH₄SO₃, 4 NaCl, 1 MgCl₂, 3 KCl, 9 EGTA and 8 HEPES (pH 7.3, adjusted with CsOH). TTX and cesium were used to block Na⁺ and K⁺ channel currents, respectively. For characterization of mEPSCs, neurons were patched and recorded in voltage-clamp mode at −80 mV. For I-V curves, neurons were held at −70 mV and stepped from −100 mV to +60 mV in regular increments. At each potential, neurons were sequentially perfused with (i) ERB for 15 sec; (ii) ERB with 50 μM NMDA or 100 μM kainate for 10 sec; and (iii) ERB for 35 sec to restore a baseline response. Perfusions were conducted using a three barrel Fast Step system (Warner Instruments, Hamden, Conn.). I-V responses were determined by subtracting the current measured during the initial ERB perfusion from peak currents during administration of agonist. Currents were re-measured during the washout step and compared to the baseline readings to confirm that the response was fully reversible and verify the integrity of the patch. For measurement of Mg²⁺-free ERB NMDAR currents, the perfusate was formulated as above, but without Mg²⁺. For AMPAR I-V curves, Kainate (100 μM; KA) was used as a non-desensitizing agonist of AMPAR channels to avoid neurotoxicity induced by simultaneous application of AMPA and cyclothioheximide.

### Immunofluorescence and Scanning Electron Microscopy

ESNs on 18-mm coverslips were incubated in NBA supplemented with vehicle or described concentrations of glutamate for indicated durations. For immunocytochemistry, neurons treated with vehicle or 12.5 μM glutamate were fixed in 4.0% paraformaldehyde in PBS (Life Technologies) at 2, 6, or 24 h, permeabilized in 0.1% saponin (Sigma), blocked with 3% BSA (Sigma), and probed with anti-NR2A/B, anti-TAU antibody, anti-MAP2 antibody (Synaptic Systems) or anti-GluR1 antibody (Epitomics Burlingame, Calif.). Proteins were visualized with Alexa Flour-labeled goat anti-mouse, anti-rabbit, and anti-guinea pig antibodies. Coverslips were mounted onto glass slides with Prolong Gold anti-fade reagent containing DAPI (Life Technologies). Images were collected on a Zeiss LSM-700 confocal microscope with a 40× objective. Intensities were enhanced after image capture to visualize neurite integrity. Quantification of neuronal fragmentation and varicosity formation was done with NIH ImageJ. For scanning electron microscopy (SEM), neurons exposed to 200 μM glutamate or vehicle for 6 h were fixed for 30 min with buffered 1.6% paraformaldehyde/2.5% glutaraldehyde. Following fixation, samples were osmicated and processed by standard dehydration protocol to critical point drying. Dried samples were ion beam-coated with gold/palladium and imaged using a JEOL JSM-7401F field emission scanning electron microscope.

### Endpoint Evaluation of DNA Condensation and Membrane Disruption

ESNs plated on 18-mm coverslips were washed and stained with 5 μg/mL Hoechst and 5 μg/mL PI in NBA for 10 and 5 min, respectively, at 37° C. After staining, the ESNs were washed, fixed in 4.0% paraformaldehyde PBS pH 7.4, and mounted onto glass slides with Prolong Gold antifade reagent. Coverslips were imaged on a Zeiss LSM-700 confocal microscope at 40× did 63× using manufacturer-specified laser excitation wavelengths and emission filter sets. Necrosis and apoptosis were evaluated per the following. Healthy neurons had large nuclei with a diffuse chromatin morphology colored blue by Hoechst 33342. Apoptotic nuclei exhibited condensed Hoechst-stained chromatin, while necrosis resulted in PI uptake. Glial cells (˜5-10% per cover slip) were identified by light microscopy and occluded from analyses. For quantitation of nuclear size, orthogonal measurements of nuclear diameter were made using Zen 2007, averaged, and used to calculate the two-dimensional nuclear area.

### Viability Measurements

ESNs were plated in 24-well, 48-well or 96-well dishes and treated per indicated conditions To quantify metabolic activity, PrestoBlue (Invitrogen) was added to ESNs at a final 1× concentration in fresh NBA-B27 and ESNs were incubated for an additional 45 min at 37° C. Metabolic conversion of PrestoBlue to a fluorescent product was measured with a Synergy MX plate reader (Biotek, Winooski, Vt.) using excitation of 535 nm and emission of 595 nm. To evaluate potential glial contributions to metabolic activity, wells were treated with 5 mM glutamate subsequent to measurements of metabolic activity, and fluorescence intensities were subtracted from experimental wells. Microscopic evaluation of cultures confirmed that addition of 5 mM glutamate did not induce any detectable toxicity in the rare glia, whereas all neurons exhibited PI uptake or nuclear condensation within 2 h. Using this approach, glia contributed less than 5% of the metabolic activity measured in untreated controls. Since glial contamination did not appear to significantly affect measurements of toxicity, it was not used for subsequent viability assays.

For pulsed exposures, supplements prepared in BEB or in modified BEB were added at the described concentration for 5 min, washed three times with NBA-B27 and returned to the incubator for 6 or 24 h before determining viability. EC₅₀ values were calculated by fitting the dose-response curve to a four-parameter sigmoidal model using Prism v5.04 (Graphpad Software, La Jolla, Calif.). For prophylactic screening, a cocktail of neurotrophic factors (NTFs; 10 ng/mL human neurotrophin-3 [NT3], 10 ng/mL human brain-derived neurotrophic factor [BDNF], 10 ng/mL rat glial-derived neurotrophic factor [GDNF] and 25 nWmL rat ciliary neurotrophic factor [CNTF]; R&D Systems, Minneapolis Minn.) was added 18 h prior to and concurrent with the addition of glutamate.

### RNA Analyses

RNA was extracted from ESNs using the RNeasy Mini Kit (Qiagen, Germantown, Md.) and RNA quality was assessed using the NanoDrop 2000c UV-Vis spectrophotometer (Thermo Scientific). Expression profiling using next-generation sequencing (RNAseq) was conducted. Reads were assembled, annotated and screened for differential expression of Gria2 isoforms using the Seqman Pro software package (DNAStar, Inc, Madison, Wis.). Library sizes were normalized using the DESeq normalizedCounts function and normalized reads are presented as pseudocounts.

For quantitative, real-time reverse-transcription PCR (QPCR), cDNA was synthesized using the RT² First Strand Kit (SaBioxiences, Frederick, Md.) according to manufacturer's protocol). QPCR was performed using a BioRad-CFX 96-well Real-Time System with iQ SYBRGreen Supermix in a PTC-100 MJ Research thermal cycler (Waltham, Mass.). Primer sequences were from the Harvard University Medical School Primer Bank. PCR conditions were 95° C. for 3 min, followed by 39 cycles of 95° C. for 10 sec, 54° C. for 10 sec, and 72° C. for 30 sec, followed by a melt curve from 54 to 95° C. in 0.5° C. increments. The ΔC_{T} for control and treated samples were normalized to β3-tubulin to determine the normalized log₂ fold change.

### Statistics

BoNT and LTX EC₅₀ values were calculated using a four-parameter sigma model from average dose response values determined from densitometry of western blot images and presented as median values with 95% confidence intervals (C.I.). Multiple comparisons were performed using one-way analysis of variance (ANOVA) to determine significance. Differences among means were determined and calculated with the Student's t-test. * indicates a P<0.05. ** indicates a P<0.01. *** indicates a P<0.001. IPA and DAVID generated the p-values adjusted for multiple hypotheses.

In some instances, statistical significance among means was determined utilizing repeated-measures. ANOVA and P values were calculated with the Tukey's post-hoc test. For binary comparisons of means, the Student's t-test was used. Unless otherwise stated, all quantitative data are presented as mean plus/minus one standard deviation, with the following markers of statistical significance: * indicates a P<0.05; ** indicates a P<0.01; ′ indicates a P<0.001.

All toxin concentrations are based on lots of toxin with specific activity of 2.2 mouse LD50/mg.

### Example 2

### Suspension-Adapted ESCs Remain Pluripotent and Mitotically Active

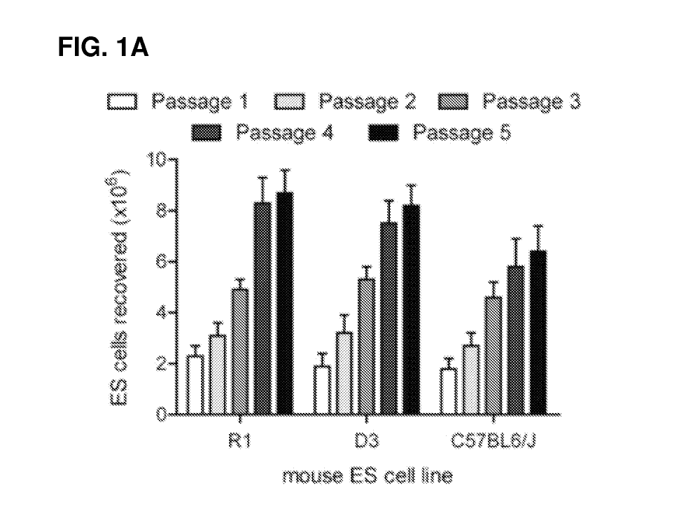

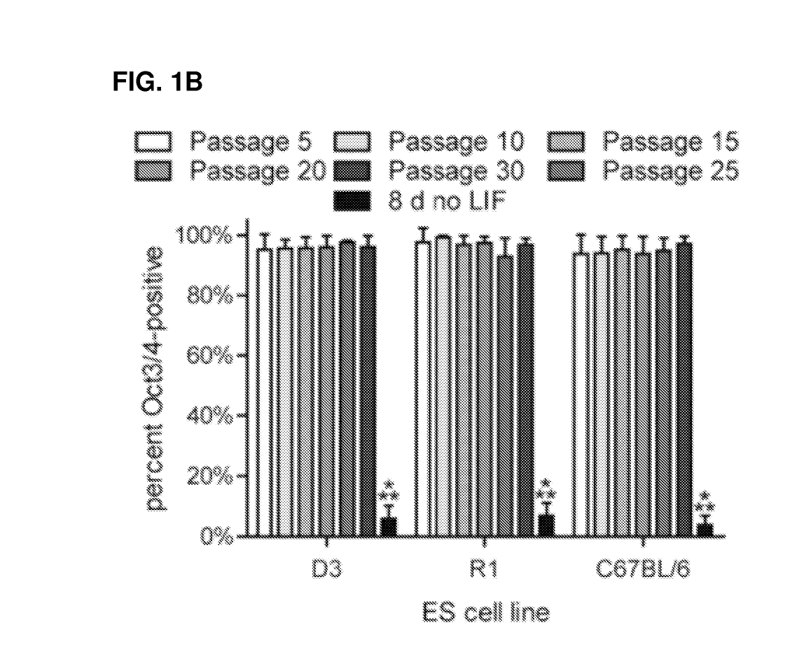

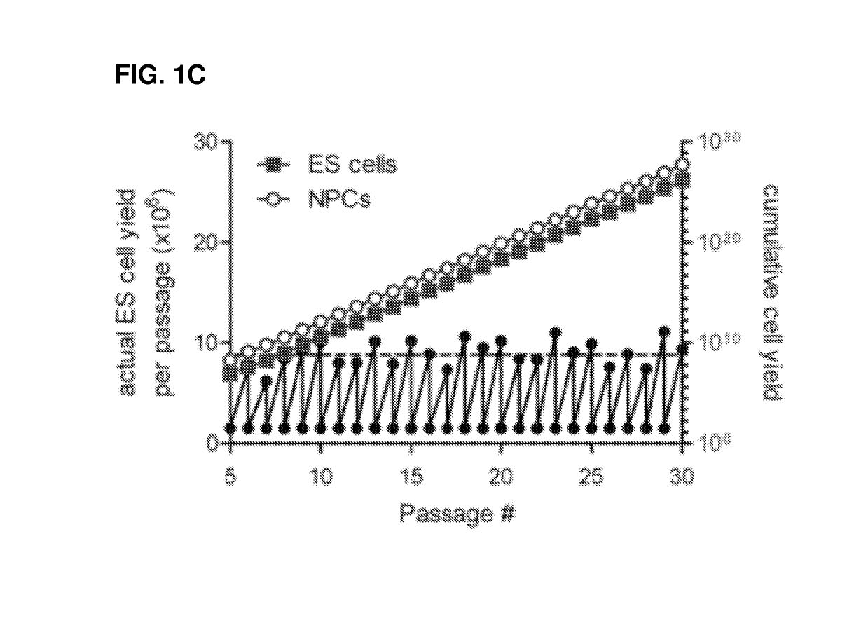

ESCs are traditionally maintained on feeder cells in the presence of LIF and fetal calf serum to preserve germline competency. To adapt ESC lines to feeder cell-free suspension culture, R1, D3 and C57BL/6 ESCs that had been co-cultured with mitotically inactivated mouse embryonic fibroblasts were dissociated and cultivated in bacterial dishes until suspended aggregates developed. Mitotic rates of the suspension-adapted ESCs stabilized by five passages (FIG. 1A) and mean doubling times were 18.7±2.4 h, 19.2±2.1 h and 23.8±1.9 h over 30 passages (R1, D3 and C57BL/6, respectively). Expression of the pluripotency marker Oct3/4 did not vary through 30 passages, whereas withdrawal of LIF resulted in a loss of Oct3/4 immunoreactivity within 8 d (FIG. 1B). Theoretical estimates of cumulative yield over 25 passages from a single dish were 10²⁶ ESCs (FIG. 1C).

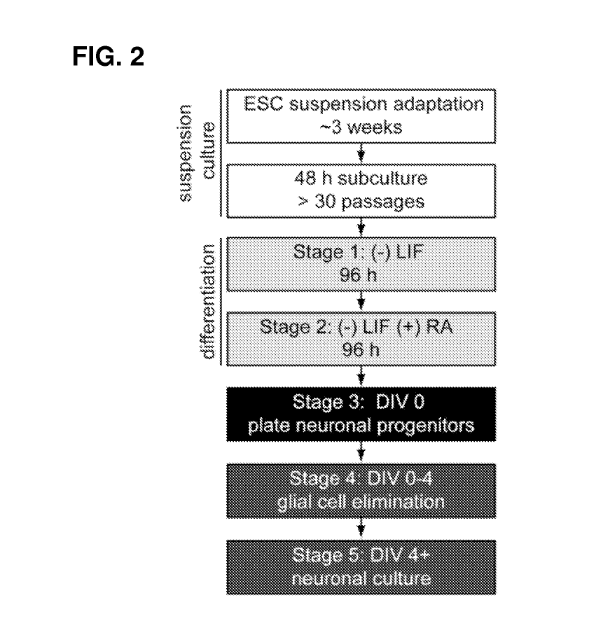

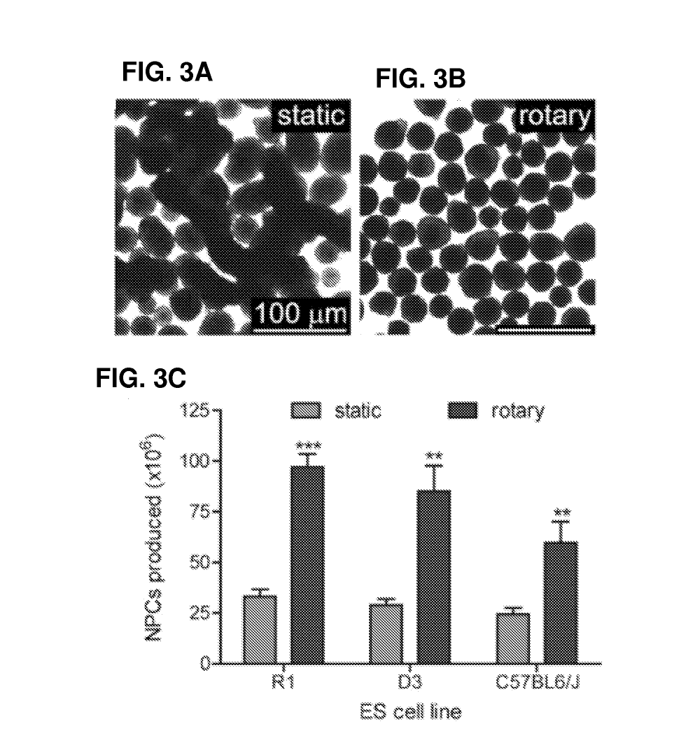

Suspension-adapted ESCs were differentiated into neurons using a modified 4/4 protocol, with increased RA concentrations and incubation under rotary conditions in low-attachment dishes (FIG. 2). Initially it was observed that differentiation under static conditions resulted in large, agglomerated complexes by DIV 0 (FIG. 3A). Hypothesizing that agglomeration might limit recovery of NPCs at DIV 0, NPC yields were compared between static differentiation conditions versus differentiation on a rotary shaker at 45 RPM. Rotary conditions eliminated agglomeration and increased average yield by 290% to 97×10⁶ NPCs per 10-cm dish (FIGS. 3A and 3B).

No differences were observed in NPC yield between 5-30 passages in any ESC line. In one instance, an R1 culture maintained for 65 passages produced 87×10⁶ NPCs, which underwent normal neuronal development, indicating that extended periods in suspension culture do not interfere with neurogenic competence.

### Example 3

### Differentiated Cells Express Transcriptional, Morphological and Immunological Markers of Neurogenesis

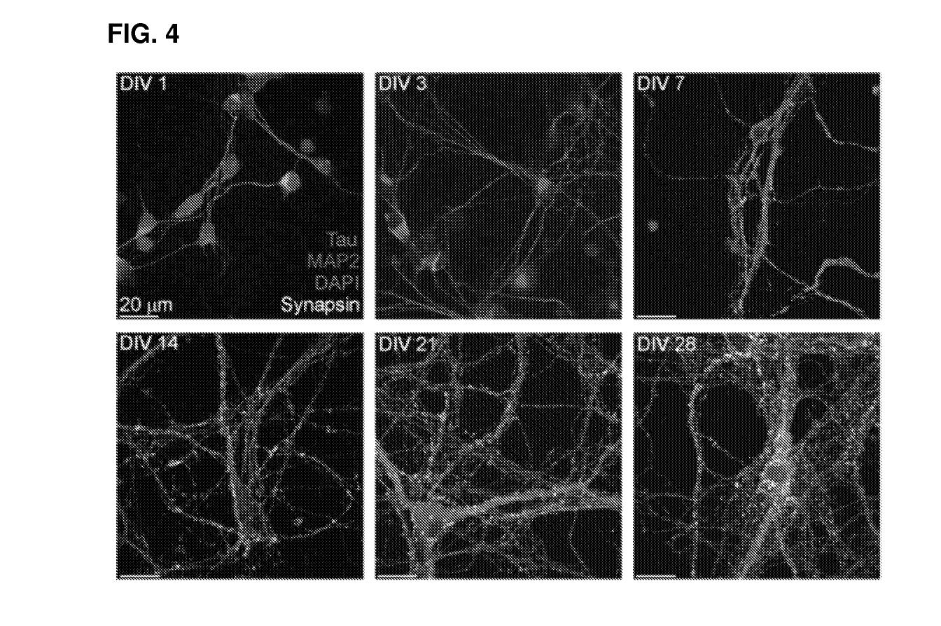

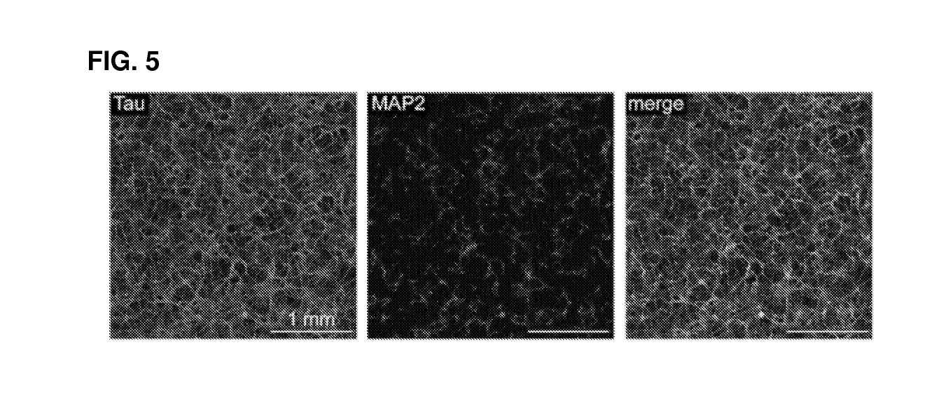

Neuronal maturation was characterized between DIV 1-28 using the dendritic marker MAP2, the axonal marker MAP-tau and the pre-synaptic marker synapsin-1 (FIG. 4). Although MAP2 was uniformly present at DIV 1, MAP-tau expression was not widely observed in the majority of neurons until DIV 3. Axonal arborization increased extensively between DIV 3-14, while dendritic extension occurred predominantly after DIV 14 (FIG. 4). Weak synapsin puncta were widely dispersed along axons at DIV 7, primarily in the absence of proximal dendrites, but as of DIV 14, synapsin-1 staining accumulated at axodendritic interfaces. By DIV 21, an extensive “lawn” of neurites was apparent, with robust axonal arborization and elongated dendrites appearing in close proximity to single or fasciculated axons (FIGS. 4 and 5). Greater than 99% of surviving cells expressed neuron-specific markers at DIV 7, and less than one GFAP⁺ glial cell was observed per mm² at DIV 21 (averaged across 25 mm²). Glial cells that did survive were most often observed at regions of high neuron density, suggesting that a supportive microenvironment (e.g., cell-cell contact) may be permissive for glial persistence in the absence of serum.

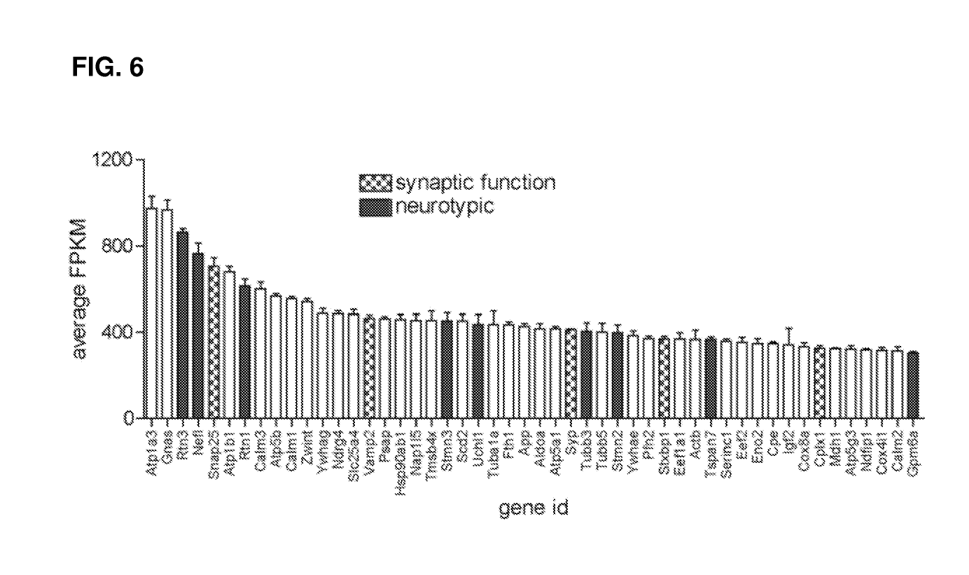

A presumptive neuronal phenotype was further evaluated by expression profiling at DIV 14. An average of 9,963±42 nuclear mRNA transcripts were detected at a single copy or higher, of which about 1,800 were present in high abundance (>30 FPKM). Overall, ESNs expressed a broad range of neurotypic genes, and 28% of the most abundant transcripts coded for neuron-specific proteins (FIG. 6A). Gene expression was highly enriched for a neuronal phenotype and strongly associated with neuron-specific canonical pathways and functions (Tables 1-3). ESNs expressed high levels of glutamatergic markers (vGluT2; 100.8 FPKM), with low-to-moderate levels of GABAergic markers (13.9 FPKM and less) and virtually no markers (<0.4 FPKM) of cholinergic, serotonergic, dopaminergic or motor neuron differentiation. A variety of neurotransmitter receptors were expressed, including those for glycine, GABA, acetylcholine (muscarinic and nicotinic) and glutamate (metabotropic and ionotropic). ESNs also expressed transcripts essential for synaptic activity and electrochemical signal propagation, including subunits of the neuronal N- and P/Q-type voltage-dependent Ca²⁺ channels (VDCCs), SNARE proteins, synaptic vesicle-associated proteins, Na⁺/K⁺ pumps, and a large number of voltage-gated Na⁺, K⁺ and Cl⁻ channels.

TABLE 1
Ingenuity Pathway Analysis (IPA) analysis of over-
represented canonical pathways, indicating p-values and the
number of mapped and identified transcripts within each pathway.
Canonical pathways

| Function | p-value | Ratio of enrichment |
|---|---|---|
| Axonal guidance signaling | 1.95 × 10⁻¹⁶ | 347/433 |
| Ephrin receptor signaling | 5.69 × 10⁻¹⁰ | 165/200 |
| Reelin signaling in neurons | 1.37 × 10⁻⁷ | 78/82 |
| Synaptic long-term | 1.17 × 10⁻⁵ | 96/114 |
| potentiation | | |
| Synaptic long-term | 1.17 × 10⁻⁵ | 118/147 |
| depression | | |
| Semaphoring signaling in | 5.98 × 10⁻⁵ | 51/52 |
| neurons | | |
| Glutamate receptors | 8.55 × 10⁻⁴ | 54/69 |
| signaling | | |

TABLE 2
IPA analysis of over-represented gene functions, indicating number
of mapped and identified trascripts associated with each function.
Gene Function Enrichment

| Function | p-value | # genes |
|---|---|---|
| Synaptic transmission | 4.90 × 10⁻¹⁵ | 271 |
| Development of brain | 7.59 × 10⁻²⁷ | 528 |
| Neuro-transmission | 4.35 × 10⁻¹⁷ | 303 |
| Neuritogenesis | 1.25 × 10⁻¹⁵ | 369 |
| Neuron guidance | 1.18 × 10⁻¹⁰ | 130 |
| Synaptogenesis | 1.18 × 10⁻¹⁰ | 130 |
| axonogenesis | 1.24 × 10⁻⁹ | 137 |

TABLE 3
Tissue-specific gene enrichment from 7939 mapped
and identified transcripts wit FPKM > 3.0 using IPA.
Percent Tissue Enrichment

| | IPA (7939) | DAVID (top 3000) | |
|---|---|---|---|
| Tissue | % | % | p-value |
| Nervous system | 91.6 | 59.3 | 1.5 × 10⁻¹⁵² |
| Lung | 71.8 | 8.3 | 1.9 × 10⁻⁴ |
| Thymus | 69.5 | 17.8 | 3.0 × 10⁻¹⁹ |
| Uterus | 66.0 | nf | nf |
| Skeletal muscle | 63.3 | 0.5 | 3.9 × 10⁻² |
| Liver | 61.8 | 25.9 | 7.2 × 10⁻² |
| Pancreas | 38.6 | 5.1 | 4.9 × 10⁻⁹ |

nf-not found.

### Example 4

### Depolarizing Stimuli Evoke Reversible Ca2+ Uptake

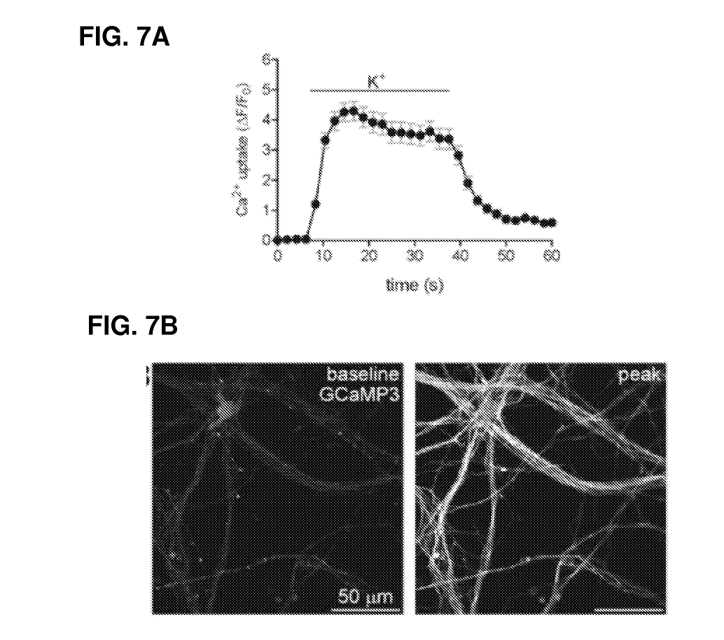

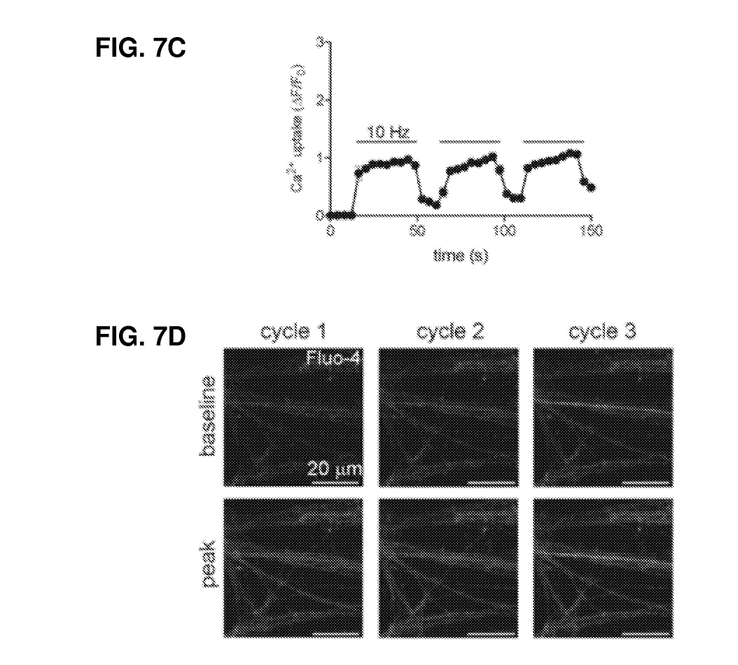

The expression of gated ion channels and pumps important in maintaining and altering membrane polarity suggested that application of depolarizing stimuli might elicit Ca²⁺ uptake. The plasma membrane potential was altered using indirect (elevated K⁺) and direct (three cycles of electrical field stimulation, 300 pulses per cycle at 10 Hz) methods and found that both methods of depolarization elicited reversible Ca²⁺ influxes (FIG. 7). The functional verification of Ca²⁺ uptake in response to classical depolarizing stimuli confirms intracellular recordings and neurotransmitter release assays, and indicates that ESNs may be sensitive to neurotoxic stimuli whose mechanism of action involves dysregulation of electrochemical signal propagation.

### Example 5

### ESNs are Sensitive to Glutamatergic Excitotoxicity

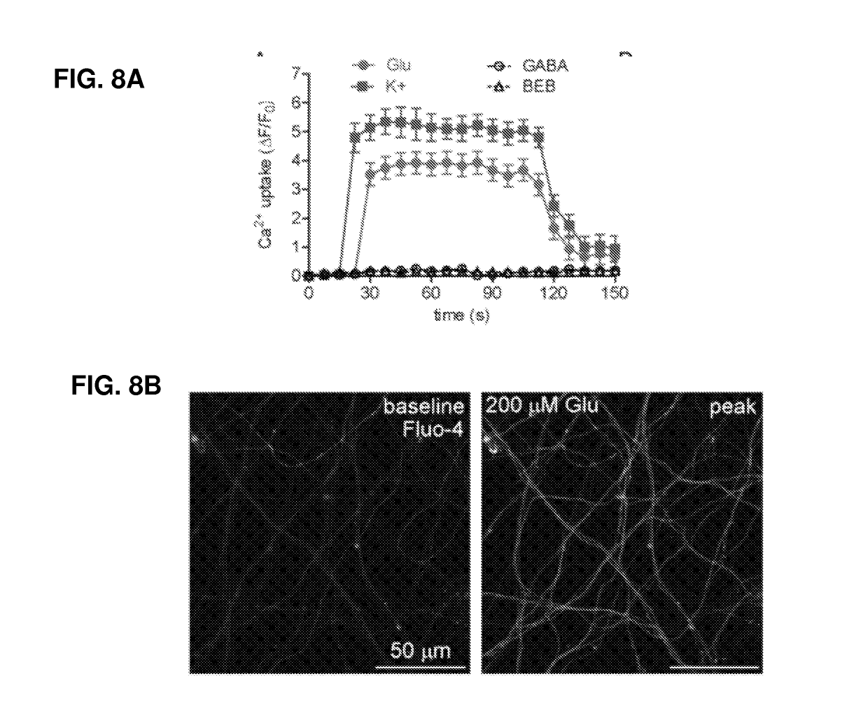

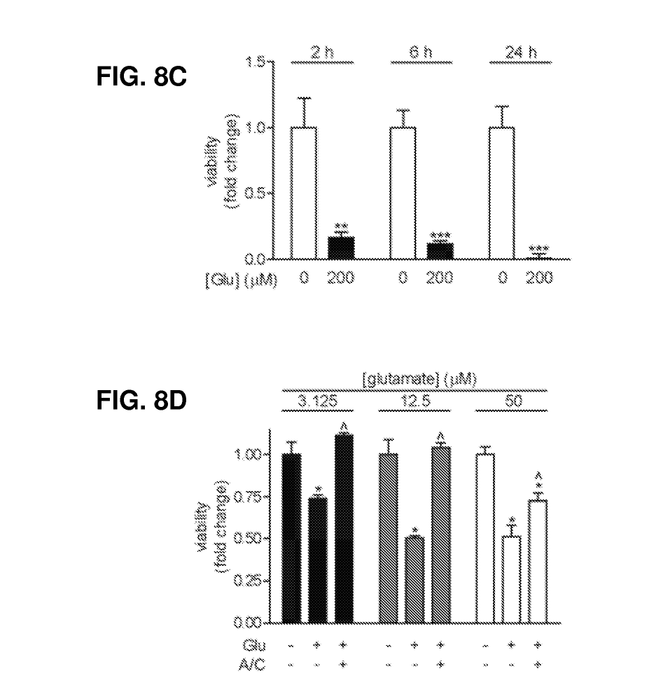

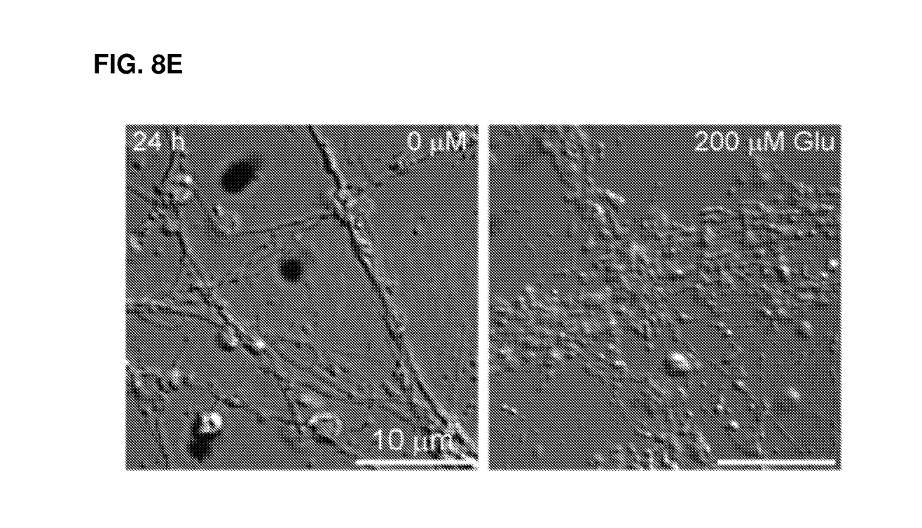

Glutamatergic excitotoxicity has been attributed to the pathologic internalization of Ca²⁺ through post-synaptic NMDA receptors, compounded by activation of VDCCs in response to excitatory post-synaptic currents (EPSCs) from AMPA and KA receptors. In primary neuron cultures, neurotoxicity has been reported over a wide range of glutamate (glu) doses and exposure durations. ESNs express transcripts for NMDA, KA and AMPA receptor subunits and the NMDA-associated protein Grina at high levels, indicating that ESNs may be functionally sensitive to glu treatment. Treatment of ESNs with 200 μM glu resulted in acute levels of Ca²⁺ uptake similar to those caused by K⁺, whereas the inhibitory neurotransmitter GABA or vehicle controls had no effect (FIGS. 8A and 8B). Glu treatment resulted in significant time-(2-24 h; FIG. 8C) and dose-dependent (3.125-200 μM; FIG. 8D) toxicity, further confirmed by morphological evidence of neurite degeneration 24 h after a 200 μM treatment (FIG. 8E). Co-administration of the GluR antagonists APV and CNQX afforded complete protection against toxicity after a 2 h exposure to 3.125 and 12.5 μM glu, and 50% protection against 50 μM glu. These results show that ESNs undergo a time- and dose-dependent glu toxicity that is mediated by ionotropic GluRs.

### Example 6

### Neuron Viability and Ca2+ Influx Following Exposure to Alpha-latrotoxin (LTX)

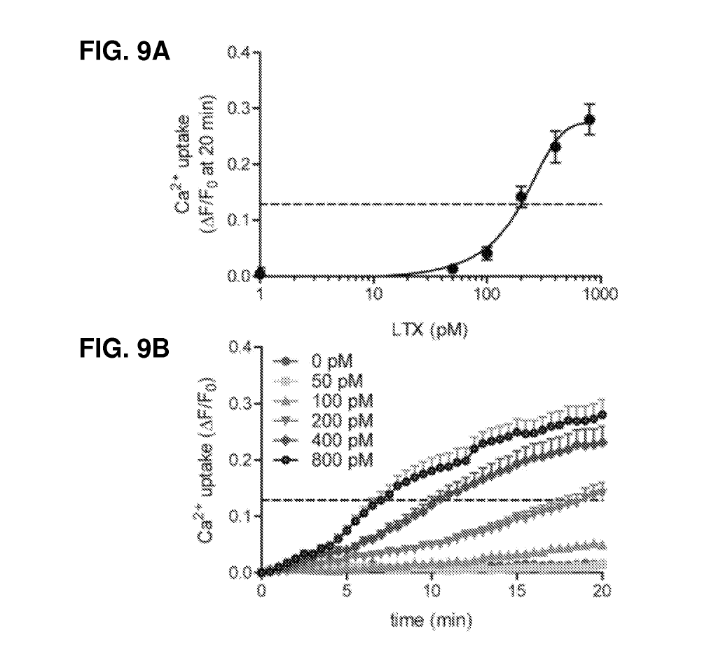

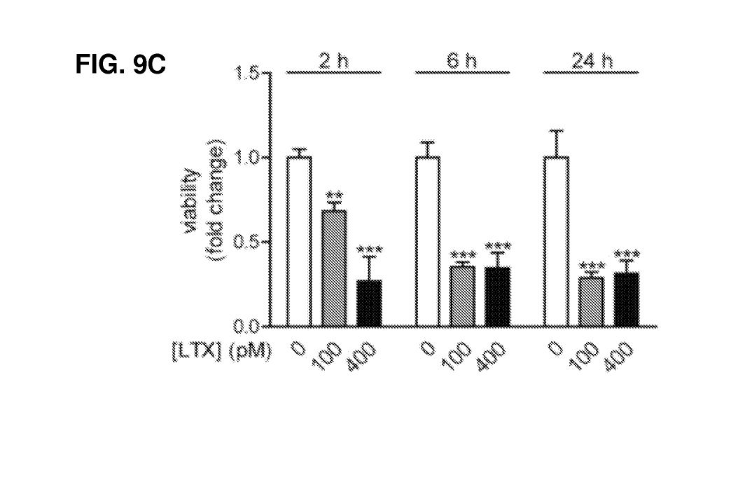

Unlike glu exposure, which induces EPSCs in postsynaptic compartments, LTX forms Ca²⁺-permissive pores in the presynaptic membrane that result in fulminant neurotransmitter release and activation of non-synaptic Ca²⁺-sensitive intracellular pathways. ESNs express transcripts of known LTX receptors (PTPRS, neurexin 1-3 and latrophilin 1-3), and LTX treatment of ESNs evokes unregulated Ca²⁺ influx, followed by morphological and biochemical indicators of neurotoxicity. To better characterize LTX toxicity, we evaluated dose-dependent relationships between LTX, onset and magnitude of Ca²⁺ uptake and neuron cell death. The EC₅₀ for Ca²⁺ uptake at 20 min was determined to be 174.9 pM (95% C.I. [68.5, 281.3]; FIG. 9A), with positive correlations between dose, onset and magnitude of Ca²⁺ uptake (FIG. 9B). There was also a strong correlation between LTX dose and inhibition of metabolic activity after a 30 min exposure (FIG. 9C). These findings illustrate the compatibility of ESNs with moderate-throughput screening approaches, confirm that ESN sensitivity and response to LTX are similar to those of primary neurons, and demonstrate that ESNs offer a novel platform with which to screen for therapeutics that prevent LTX toxicity using multi-well formats.

### Example 7

### ESNs are a Biologically Relevant Model of BoNT Intoxication

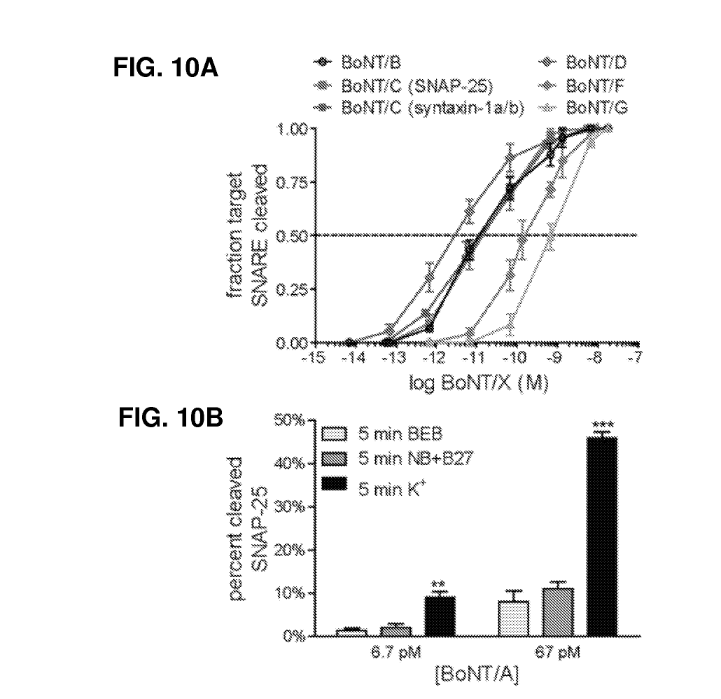

ESNs strongly express transcripts and protein for proteolytic targets (SNAP25, VAMP-2 and syntaxin) of the seven BoNT serotypes and known protein receptors for presynaptic uptake (SV2 or synaptotagmin). ESNs are sensitive to femtomolar concentrations of BoNT/A and /E. To determine whether ESNs are a suitable model for other serotypes, cleavage of the target SNARE proteins was analyzed after a 24 h exposure to BoNTs/B, /C, /D, IF and /G (FIG. 10A). ESNs exhibited similar or improved sensitivities to all BoNT serotypes tested compared to primary mouse spinal cord or cerebellar granule cell neurons, and several orders of magnitude improved sensitivity over neuroblastoma cells. BoNT/C is the only holotoxin that targets multiple SNARE proteins; interestingly, intoxication of ESNs with BoNT/C resulted in cleavage of SNAP-25 as well as syntaxin-1 with roughly equivalent EC₅₀ values.

BoNT holotoxin binds to pre-synaptic receptors and is internalized via synaptic endocytosis. Following cell entry, the proteolytically active light chain is released to the synaptic compartment through pores formed by the heavy chain in the endosome membrane. BoNT uptake and activation are enhanced following intoxication under depolarizing conditions (presumably the consequence of an accelerated rate of synaptic endocytosis). A 5 min intoxication of ESNs by BoNT/A in depolarizing media increased SNAP-25 cleavage by five-fold after 24 h as compared to two different basal media (FIG. 10B).

ESNs are sensitive to all seven classical BoNT serotypes, with EC₅₀ values that are similar to those of primary neuron cultures and several orders of magnitude improved over neurogenic cell lines (Table 4).

TABLE 4
Sensitivities of primary neurons, human neuroblastomas
and ESNs to the seven classical BoNT serotypes.

| | | Primary | | |
|---|---|---|---|---|
| | Target | neurons | Neuroblastomas | ESNs |
| BoNT | SNARE | EC₅₀ | (SH-SY5Y) | EC₅₀ |
| serotype | protein | (pM) | EC₅₀ (pM) | (pM) |
| BoNT/A | SNAP-25 | 0.4^{A} | 5562 | 0.80 |
| | | 10^{B} | | |
| BoNT/B | VAMP2 | 100^{B} | 41,650 | 22.50 |
| BoNT/C* | SNAP-25 | 13^{A} | nr | 14.24 |
| BoNT/C | Syntaxin-1 | nr | nr | 7.51 |
| BoNT/D | VAMP2 | nr | 2560 | 3.09 |
| BoNT/E | SNAP-25 | 36^{A} | nr | 66.73 |
| | | 43^{B} | | |
| BoNT/F | VAMP2 | 1350^{B} | >300,000 | 157.82 |
| BoNT/G | VAMP2 | nr | nr | 689.76 |

Primary neurons values are compiled from two sources. Annotation by (A) represents 50% inhibition of neurotransmitter release in rat cerebellar neuron cultures exposed to BoNTs for 24 h (a 90% correlation was shown between neurotransmitter release and SNARE integrity) (Foran P G, et al. J Biol Chem 278: 1363-1371, 2003) whereas annotation by B represents cleavage of 50% of SNAP-25 protein in fetal mouse spinal cord cultures exposed to BoNT for 48 h (Keller J E, et al., FEBS Lett 456: 137-142, 1999). SH-SY5Y neuroblastomas values represent inhibition of 50% of noradrenalin release in SH-SY5Y human neuroblastomas cells after 72 h incubation (Purkiss J R, et al. Neurotoxicology 22: 447-453 2001). ESN values represent 50% cleavage of SNARE proteins in ESNs. nr, not reported

### Example 8

### Optimization of the Screening Model

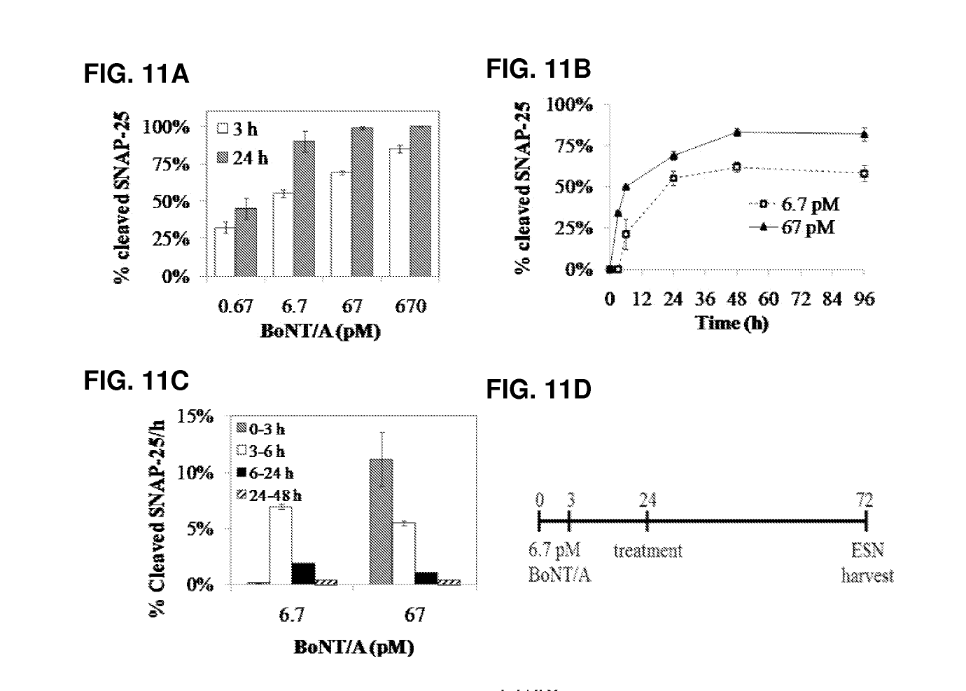

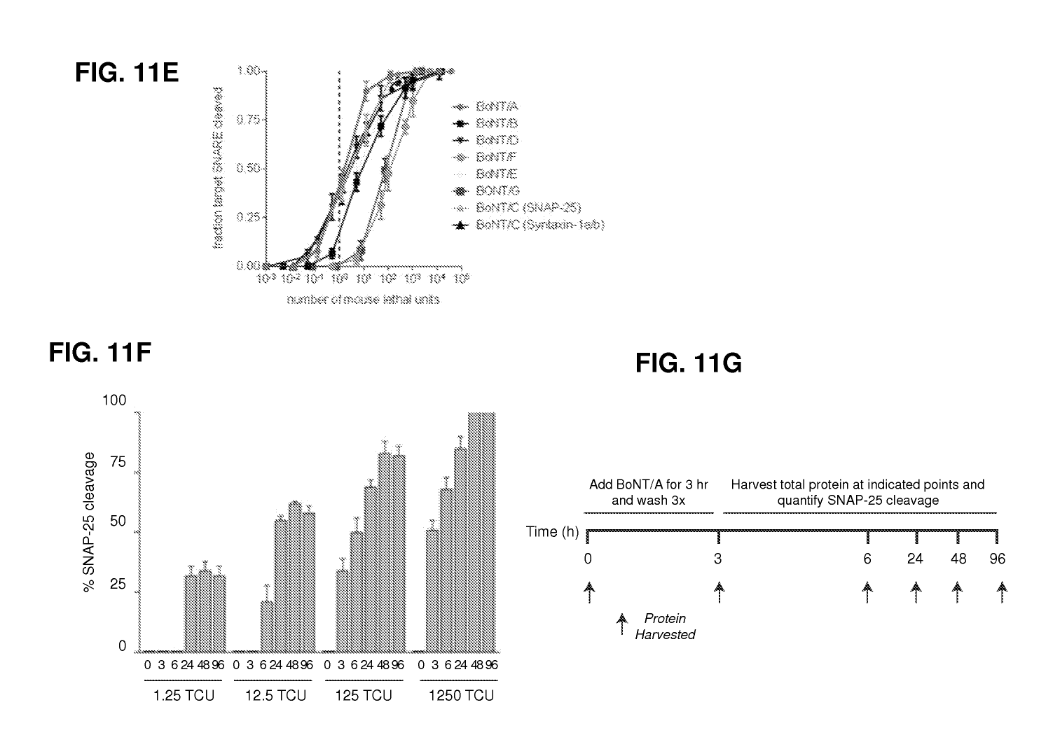

Treatment with 0.81 pM BoNT/A for 24 h results in cleavage of 50% of SNAP-25 in ESNs. To determine if higher doses for a shorter period might accelerate toxin internalization, ESNs were exposed to 0.67-670 pM BoNT/A for 3 h or 6 h and SNAP-25 cleavage was evaluated after 24 h (FIG. 11A). In comparing the percent of cleaved SNAP-25 at 24 h following either 3 or 24 h intoxication, it was found that roughly three-quarters of toxin internalization occurs within the first few hours. Since both 6.7 and 67 pM produced roughly 50% cleaved SNAP-25 after 3 h exposure, BoNT/A activity was longitudinally evaluated from 3-96 h (FIG. 11B). At each concentration the rate of SNAP-25 cleavage per hour slowed dramatically after 24 h, indicating a balance between rates of SNAP-25 synthesis and cleavage (FIG. 11C). Based on these data, a screening assay was designed in which ESNs were exposed to 6.7 pM BoNT/A for 3 h, then washed and incubated for 21 h to allow full toxin activation. Candidate therapeutics were applied at 24 h, and the recovery of full-length SNAP-25 was evaluated 48 h after therapeutic treatment (FIG. 11D). The duration of incubation following treatment was selected based on reports that approximately 2% of cellular SNAP-25 is recycled per hour.

### Example 9

### LTX Treatment of BoNT/a-Treated ESNs Restores Full-Length SNAP-25 Protein within 48 h

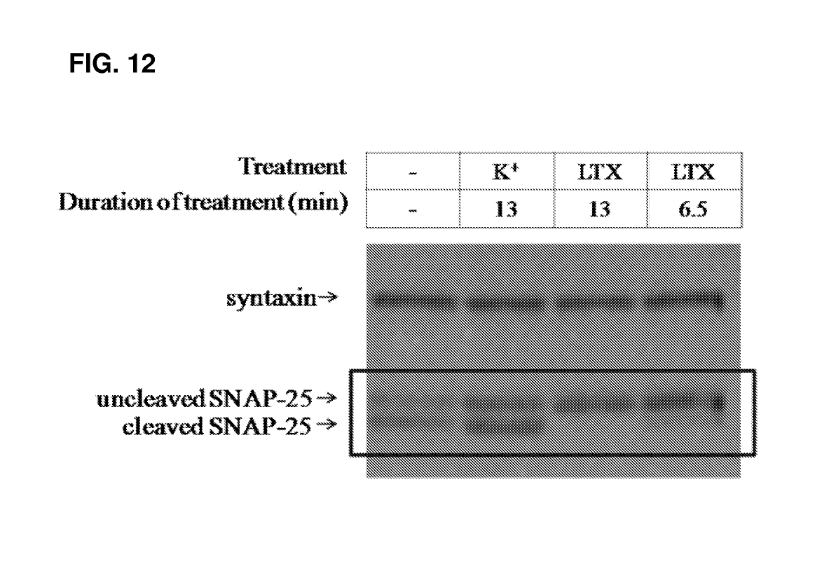

To evaluate whether LTX treatment altered light chain (LC)/A activity in ESNs, BoNT/A-intoxicated ESNs were exposed to 400 pM LTX for 6.5 or 13 min, and SNAP-25 integrity was evaluated after 48 h. As a control, ESNs were also treated with 60 mM K+ (KEB) for 1.5 min, which evokes Ca²⁺-dependent glutamate release. LTX treatment resulted in rescue of 92±4.6% and 98±1.7% (6.5 and 13 min, respectively) of full-length SNAP-25 within 48 h, whereas KEB treatment showed no difference from untreated neurons (FIG. 12). The restoration of uncleaved SNAP-25 indicates that some aspect of LTX treatment results in the inactivation or clearance of LC/A from synaptic termini. Furthermore, these data show that latrotoxin may be the active moiety in experiments demonstrating that the administration of crude homogenate from black widow spider venom glands to BoNT/A-paralyzed neuromuscular junctions dramatically accelerates recovery.

### Example 10

### LTX Treatment Results in Prolonged Ca²⁺ Internalization

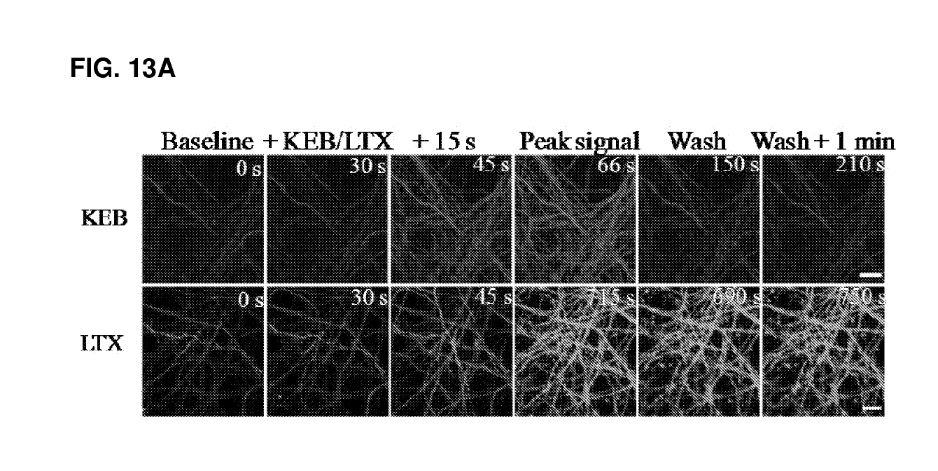

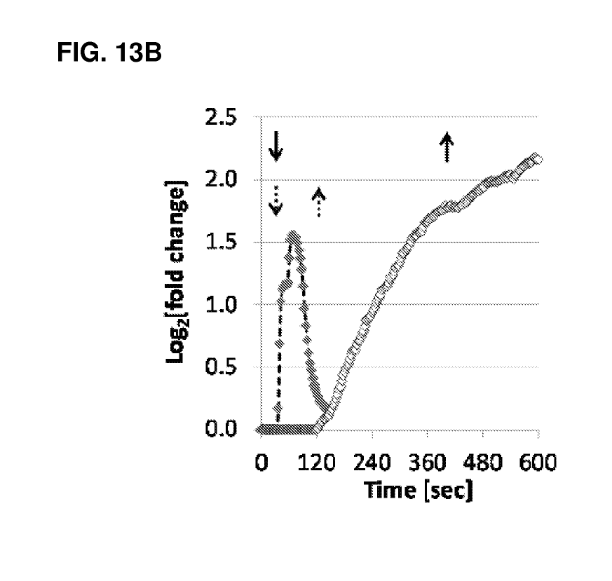

LTX treatment incurs fulminant neurotransmitter release from primary neurons, partly in response to profound levels of Ca²⁺ internalization. Since LTX treatment resulted in recovery of full-length SNAP-25 in ESNs, whereas KEB did not, we used the fluorescent intracellular calcium sensor Fluo-4 to compare the amplitude and duration of Ca²⁺ internalization evoked by these two treatments. Time-lapse confocal microscopy analysis of DIV 21 ESNs treated with KEB demonstrated an immediate rise in Fluo-4 fluorescence (FIG. 13A). Fluorescence intensity increased within 15 s of KEB treatment, remained high for about 90 s and subsided prior to washout at 2.5 min (FIG. 13B). Conversely, while ESNs treated with 400 pM LTX also showed a strong rise in Fluo-4 fluorescence, there were several key differences in the kinetics of the Ca²⁺ response. First, there was a 1.5 min delay between LTX addition and the onset of Fluo-4 fluorescence, indicating that spider toxin requires time to bind synaptic receptors and form pores within the membrane at this concentration (FIG. 13B). Unlike the self-limiting response from KEB treatment, LTX induced a steady increase in Fluo-4 signal throughout the experiment, even following rinses to remove residual toxin, demonstrating that LTX results in loss of cellular ionic homeostasis and that the KEB response was not limited by reduced levels of extracellular Ca²⁺. No apparent differences in onset, amplitude or duration were noted within the 20 min experimental window between the 6.5- and 13 min LTX treatment, nor did a 24 h intoxication with 67 pM BoNT/A prior to LTX or KEB treatment affect Ca²⁺ internalization.

### Example 11

### LTX-Treated ESNs Exhibit Evidence of Excitotoxicity that Partially Resolves Between 24-48 h

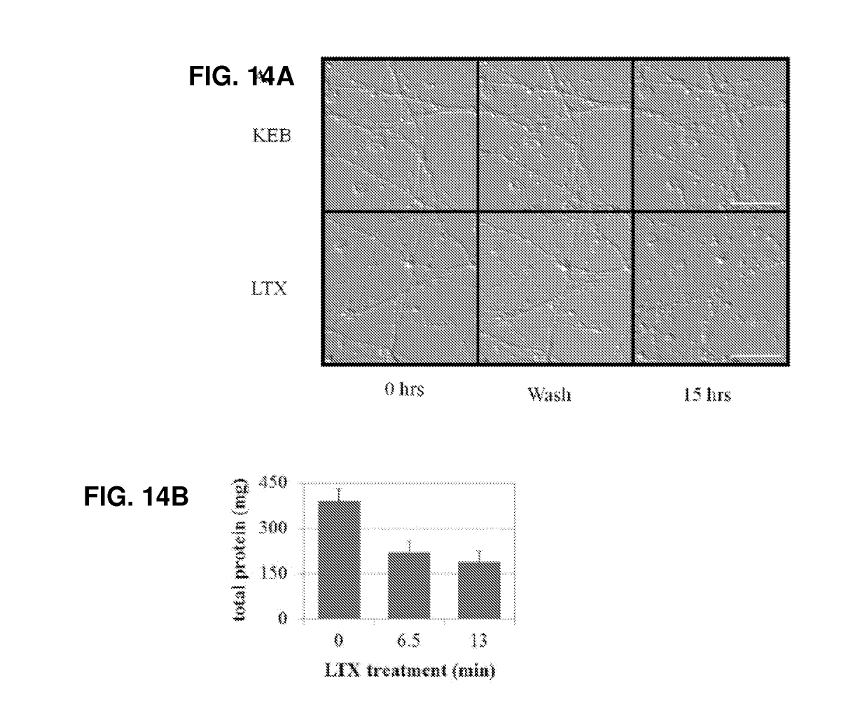

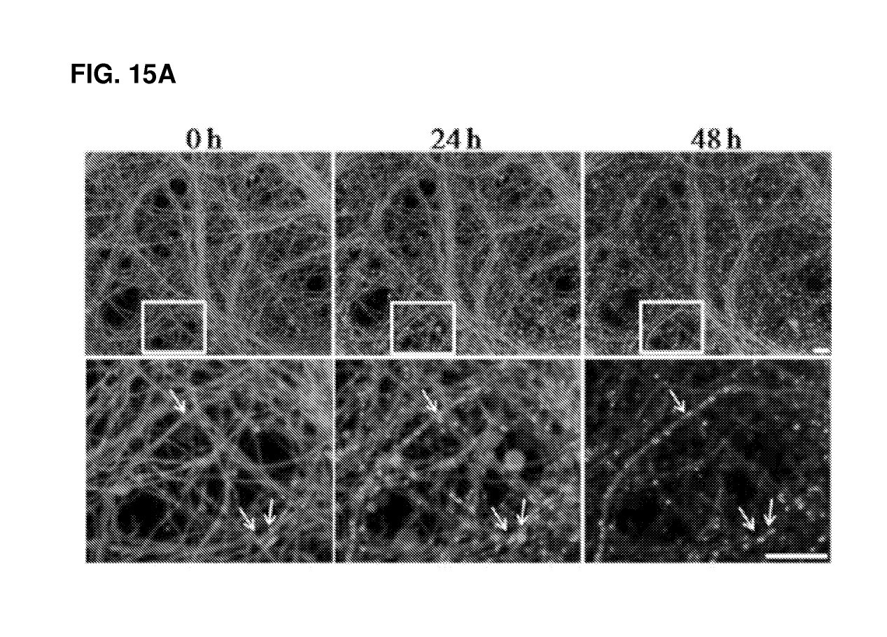

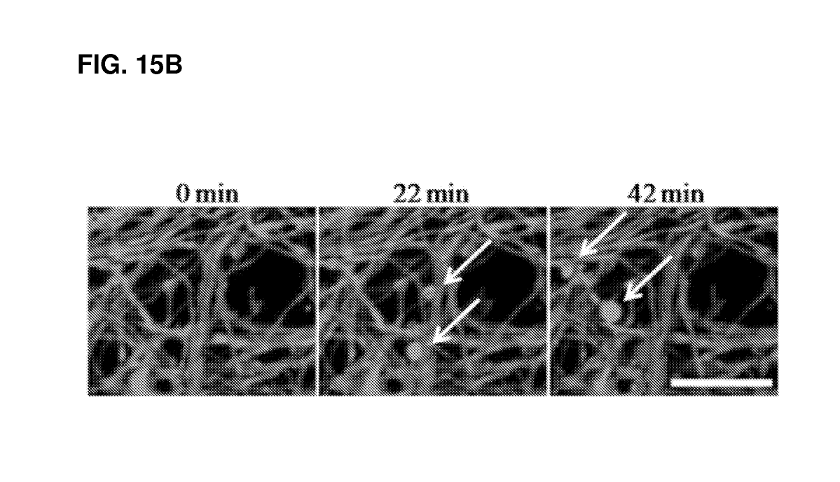

Differential interference contrast images captured 15 h after treatment indicate that cultures treated with LTX for 6.5 min have large numbers of distributed varicosities and disrupted processes, whereas cultures treated with KEB for 13 min do not (FIG. 14A). LTX treatment of ESNs decreased the total protein yield following cell lysis by 44±16% and 52±19% (6.5 and 13 min, respectively) compared to controls at 48 h after treatment (p<0.01, n=6; FIG. 14B). We used calcein green staining to compare morphological changes in ESNs during the emergent and long-term response to LTX versus KEB. These observations are in agreement with results from LTX-treated spinal cord motoneurons and cerebellar granule neurons. The development of axodendritic varicosities from existing processes was apparent within 30 min of LTX addition (FIG. 15). Although neurons remained viable 24 h and 48 h after treatment, there was an overall decrease in viability and persistent evidence of varicosities. By 48 h some of these varicosities were resolved, suggesting that neuronal regeneration was underway.

### Example 12

### Electrophysiologic Characterization of DIV 18-24 ESNs

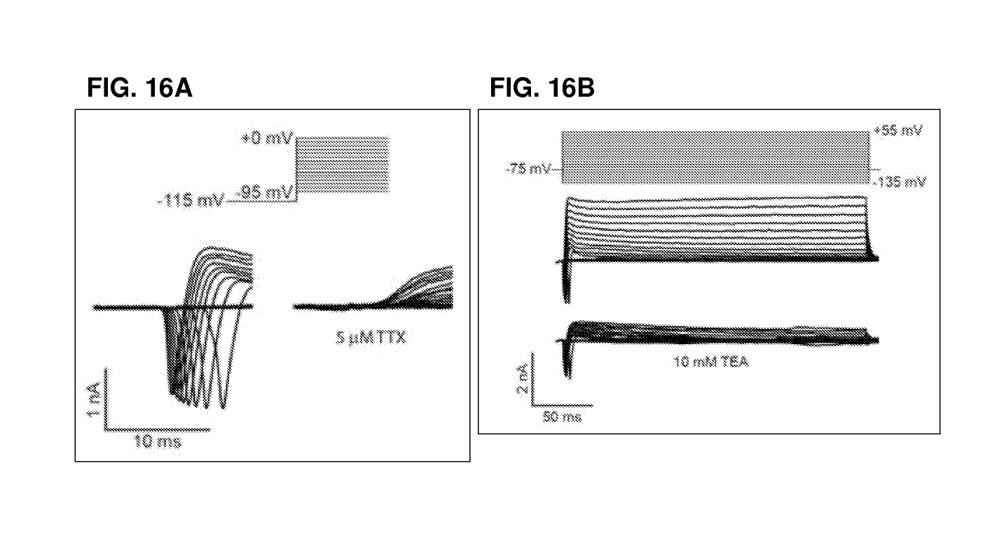

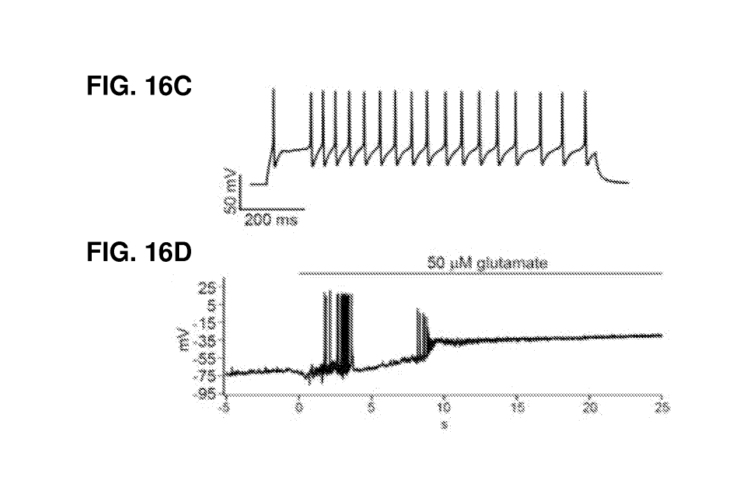

To explore the possibility that stem cell-derived glutamatergic neurons might serve as a model of excitotoxicity, a method of neuronal differentiation from feeder-cell free suspension monocultures of ESCs was adapted. Starting at eighteen days after plating (DIV 18; corresponding to 26 days after the start of differentiation), developmental stage IV/V ESNs were evaluated for neuronal activity using the whole-cell recording configuration. After adjustment for liquid junction potential, the resting membrane potential (RM P) was measured at −84.5±4.5 mV (mean±standard deviation; n=64). Voltage-clamp recordings revealed a fast-activating, fast-inactivating, TTX-sensitive inward sodium current between −45 and −55 mV (mean=48.9±4.2) (FIG. 16A) and an outward TEA-sensitive current typical of a delayed rectifier potassium conductance (FIG. 16B). Repeated overshooting action potentials were produced in response to depolarizing current pulses (FIG. 16C). Bath application of 10 μM glutamate evoked a series of action potentials lasting approximately 5-10 seconds, followed by a depolarization block that persisted until glutamate washout (n=14; FIG. 16D). Finally, inhibitory post-synaptic currents were rarely observed, consistent with transcriptomic and proteomic data that ESNs are predominantly glutamatergic.

### Example 13

### Transcriptional and Proteomic Characterization of iGluR Expression

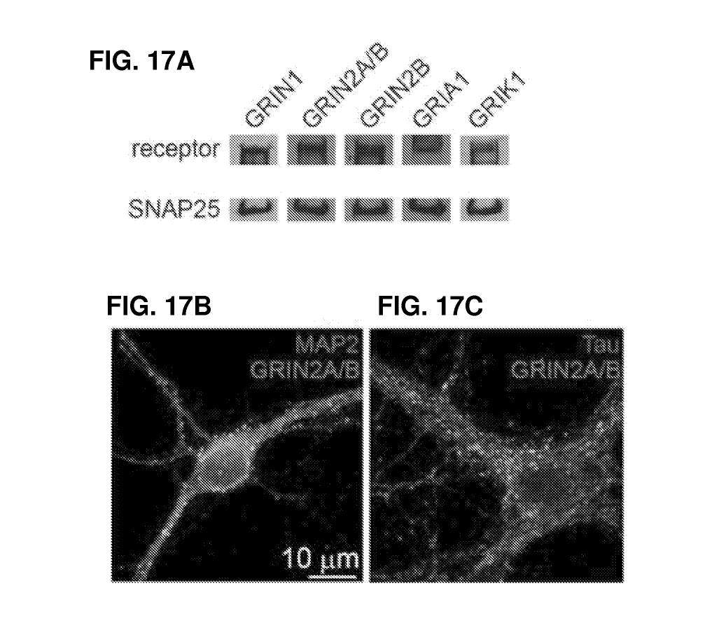

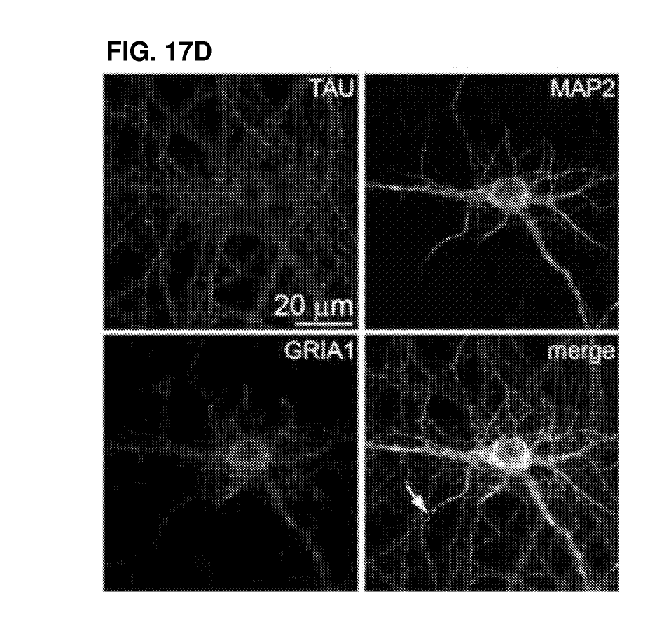

Evidence of an acute electrophysiological response to glutamate addition indicated the functional expression of ionotropic glutamate receptors (iGluRs). RNA sequencing data from a Longitudinal expression profiling experiment was screened for transcripts of iGluRs at DIV −8 (ESCs), DIV 0 (neural progenitor cells) and DIV 16 (developmental stage IV/V neurons; Table 5). The majority of subunits were either not expressed, or expressed at low levels in ESCs. In neural progenitor cells, transcripts for all AMPAR subunits were present at moderate levels while Grik5 was strongly up regulated. At DIV 16, ESNs expressed most of the subunits of NMDAR, AMPAR and KAR, with particularly high abundances of Gria1-4, Grik5 and Grin1. Single nucleotide polymorphism analysis confirmed that 98.8% of Gria2 transcripts exhibited the Q/R RNA edit that blocks AMPAR Ca²⁺ permeability (no reads were available at that position at DIV −8 or 0). The Adarb1 gene responsible for this edit was also strongly expressed at DIV 16. Immunoblot was used to confirm expression of GRIN1, GRIN2a/b, GRIA1 and GRIK1 protein (FIG. 17A) and immunofluorescence at DIV 18 revealed a punctate somatodendritic distribution for GRIN2a/b and GRIA 1, consistent with localization to post-synaptic compartments (FIG. 17B-D).

TABLE 5
RNA-seq analysis of iGluR expression as a function of neuronal differentiation
from mouse embryonic stem cells to glutametegic neurons.

| | | Embryonic stem | | Neural progenitor | | DIV 16 neurons | |
|---|---|---|---|---|---|---|---|
| | | cells (n = 4) | | cells (n = 3) | | (n = 5) | |
| iGluR class | Gene | Average | SD | Average | SD | Average | SD |
| AMPAR subunits | Gria1 | 1.6 | 1.0 | 132.4 | 42.5 | 3964.2 | 187.2 |
| | Gria2 | 2.7 | 1.4 | 328.5 | 80.8 | 7393.6 | 390.0 |
| | Gria3 | 31.1 | 5.8 | 409.8 | 27.2 | 2201.8 | 116.4 |
| | Gria4 | 88.7 | 23.2 | 291.3 | 34.7 | 6621.4 | 628.8 |
| KAR subunits | Grik1 | 0.3 | 0.5 | 150.5 | 8.3 | 771.1 | 42.6 |
| | Grik2 | 4.1 | 1.9 | 160.8 | 27.0 | 1084.0 | 64.2 |
| | Grik3 | 301.2 | 65.5 | 128.8 | 13.5 | 807.7 | 36.1 |
| | Grik4 | 16.1 | 6.9 | 104.8 | 0.8 | 641.2 | 36.2 |
| | Grik5 | 334.0 | 103.2 | 1525.6 | 171.3 | 4617.2 | 152.4 |
| NMDAR subunits | Grin1 | 313.8 | 90.6 | 70.9 | 14.7 | 12398.0 | 514.1 |
| | Grin2a | 6.0 | 1.4 | 3.7 | 1.3 | 795.9 | 67.2 |
| | Grin2b | 23.0 | 9.4 | 11.9 | 3.6 | 1577.5 | 159.2 |
| | Grin2c | 6.0 | 4.0 | 6.0 | 5.5 | 14.4 | 4.4 |
| | Grin2d | 61.4 | 11.8 | 25.2 | 3.1 | 359.7 | 26.2 |
| | Grin3b | 3.8 | 0.6 | 10.7 | 4.8 | 9.4 | 2.9 |
| | SNAP25 | 39.8 | 7.7 | 540.0 | 155.2 | 24639.3 | 1592.7 |
| | Adar1b | 558.2 | 53.3 | 376.5 | 71.0 | 3070.7 | 119.8 |

* The average pseudocount and standard deviation (SD) are presented for each iGluR subunit. Single copy is estimated to correspond to 100 pseudocounts.

### Example 14

### Functional Verification of iGluR Expression

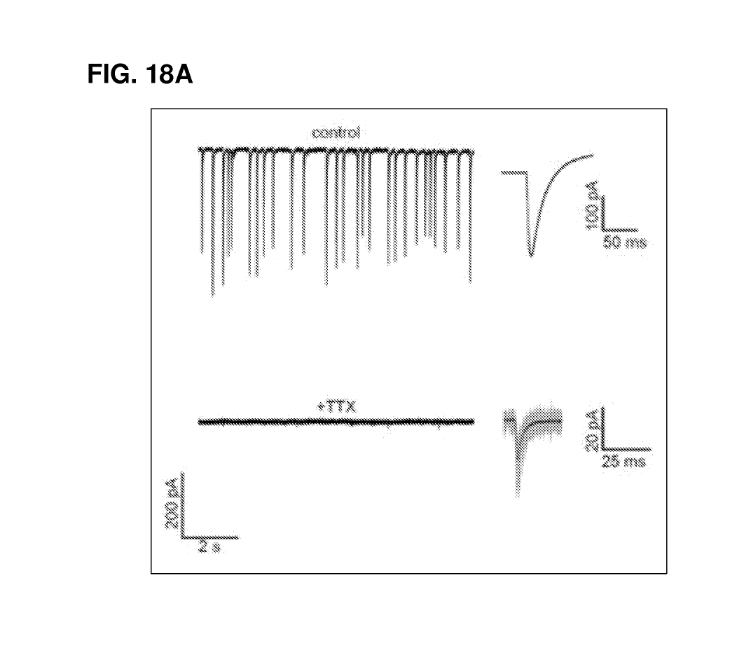

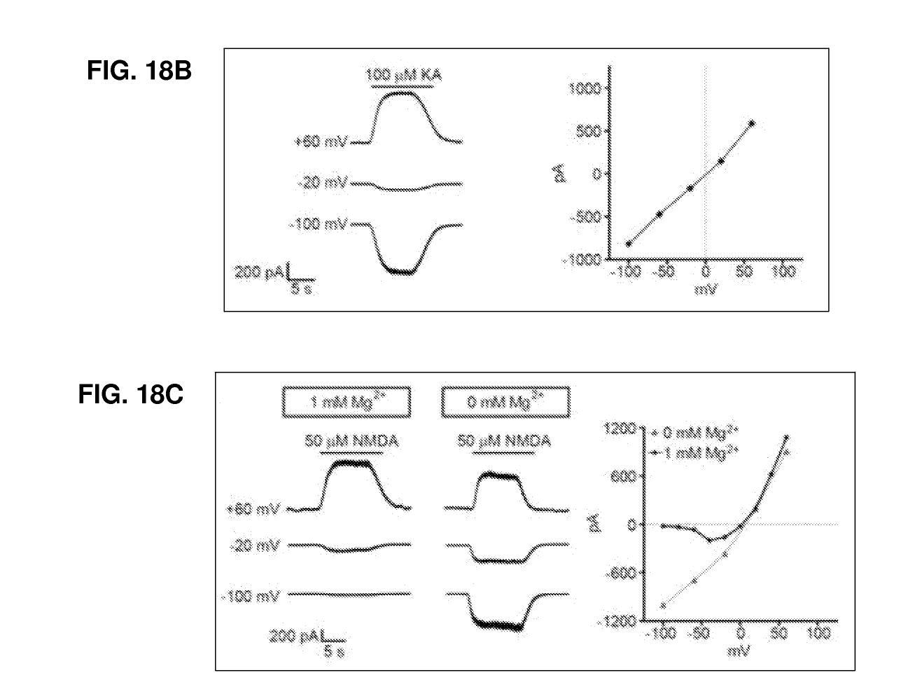

All neurons examined at DIV 18+ exhibited spontaneous excitatory post-synaptic currents (sEPSCs) at RMP (n=28; FIG. 18A). To further investigate the functional characteristics of glutamate receptor-mediated currents, the kinetics of miniature EPSCs (mEPSCs) were characterized at DIV 18-24 in the presence of TTX (5 uM) and Mg²⁺ (1 mM) at a holding potential of −80 mV (2200 events per neuron, n=12 neurons; FIG. 18A). Averaged mEPSCs had an amplitude of −18.7±1.05 pA, 10-90% rise time of 1.22±0.20 ms and width at half-amplitude of 1.73±0.09 ms. A double-exponential curve was the best fit in most circumstances, with an average fast component of 1.39±0.17 ms and slow component of 4.28±0.68 ms. The presence of a two-phase decay indicated functional expression of NMDARs and AMPARs. Current-voltage (I-V) plots were generated in whole-cell configuration to further evaluate the voltage-mediated behaviors of AMPARs and NMDARs. While AMPAR currents were linearly proportional to voltage (FIG. 18B), the NMDAR I-V curve exhibited a J-shape, consistent with a voltage-block at negative potentials (FIG. 18C). Repeating the NMDAR I-V analysis in Mg²⁺-free medium eliminated the voltage sensitivity, confirming that NMDARs exhibit the functional responses necessary for coincidence detector behavior.

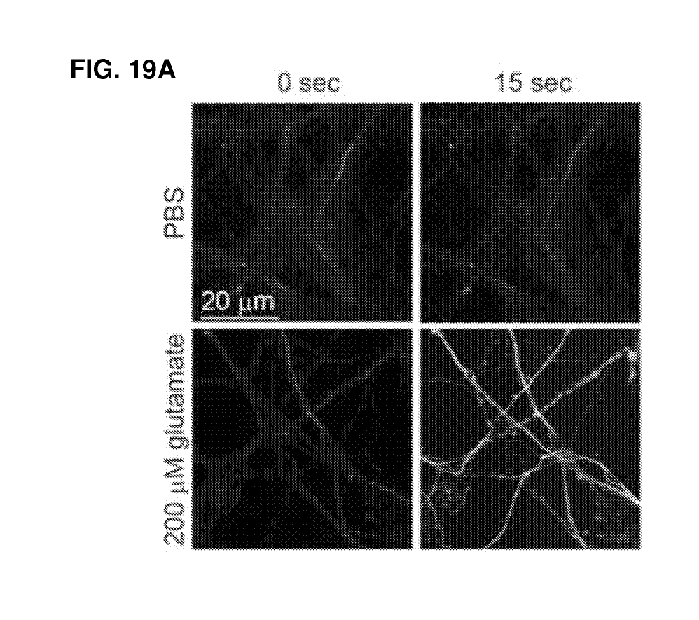

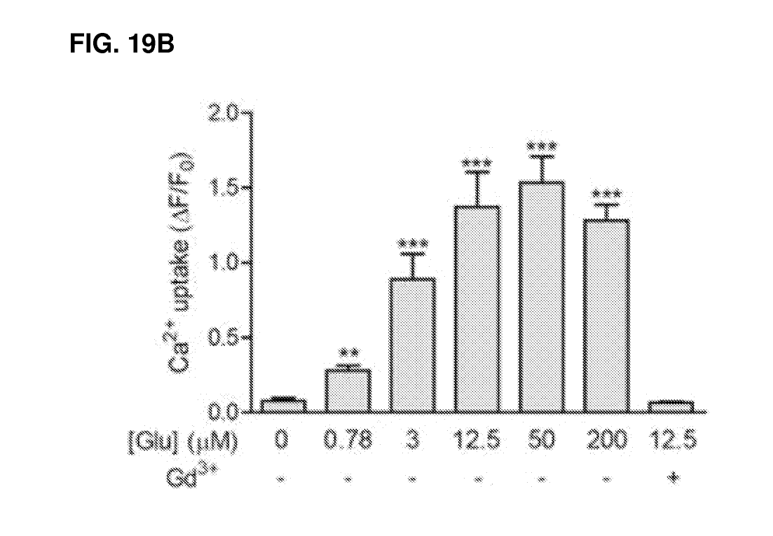

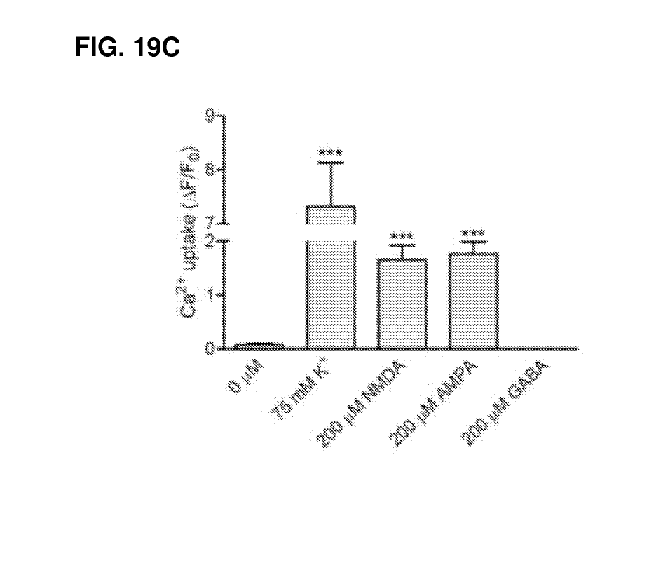

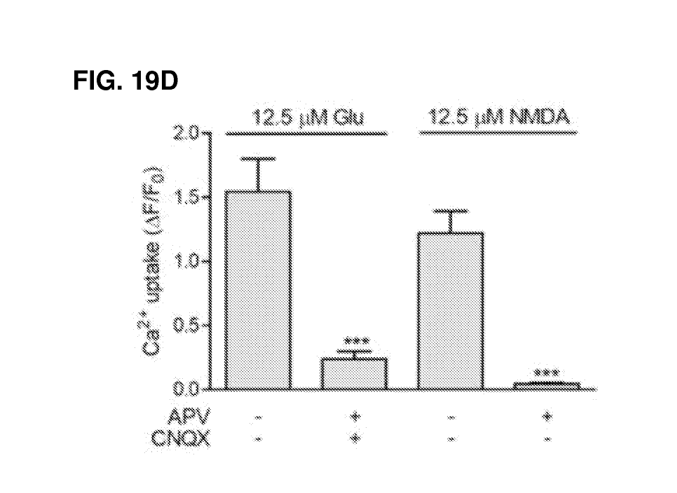

Live imaging of Ca²⁺ uptake was used to further evaluate the functional expression of iGluRs. Glutamate addition evoked a strong Ca²⁺ influx within 15 s (FIG. 19A), with a positive correlation between dose and the magnitude of fluorescence (FIG. 19B). The Ca²⁺ signal was most robust in axons suggesting that glutamate treatment was activating voltage-gated Ca²⁺ channels (VGCCs). This was confirmed by pre-treatment with the VGCC antagonist gadolinium (Gd³⁺), which eliminated over 95% of the acute Ca²⁺ signal (FIG. 19B). Acute axonal Ca²⁺ uptake was elicited by treatment with the specific iGluR agonists AMPA or NMDA, but not the inhibitory neurotransmitter GABA (FIG. 19C), and glutamate-induced Ca²⁺ uptake was efficiently blocked by pre-incubation with APV and CNQX (FIG. 19D).

### Example 15

### Acute Cytotoxicity is Mediated by NMDAR Activation in the Presence of Ca²⁺

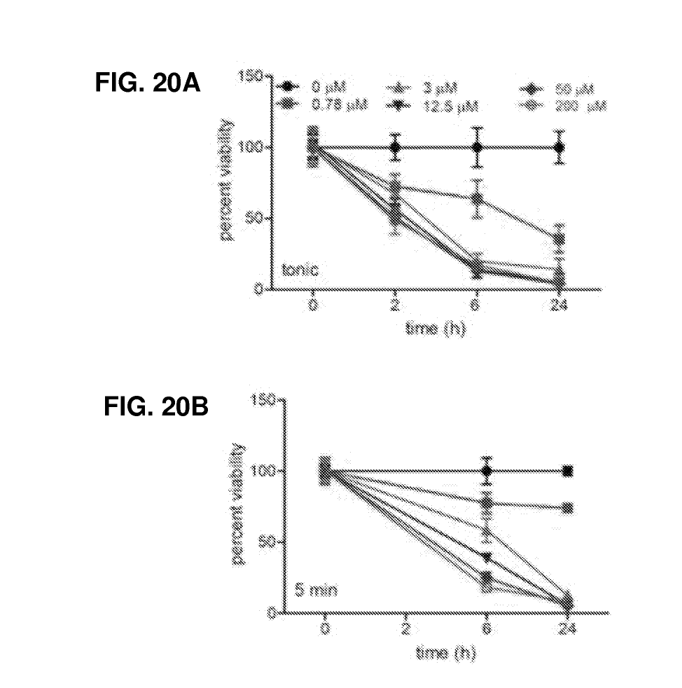

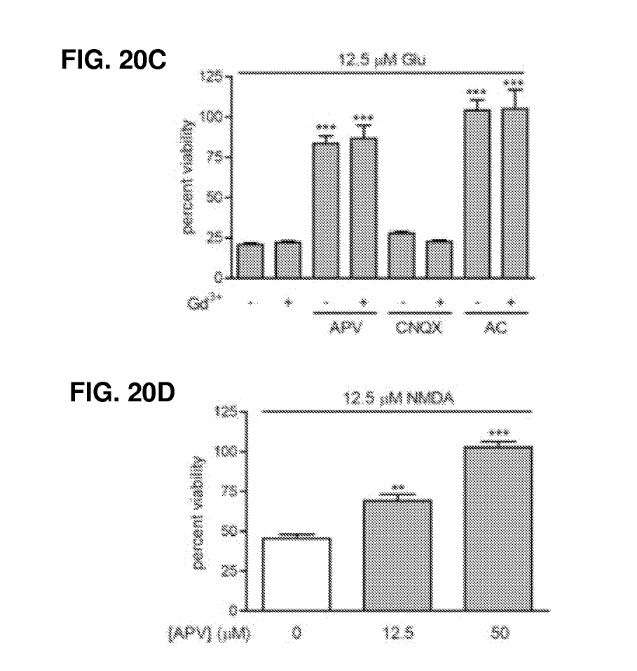

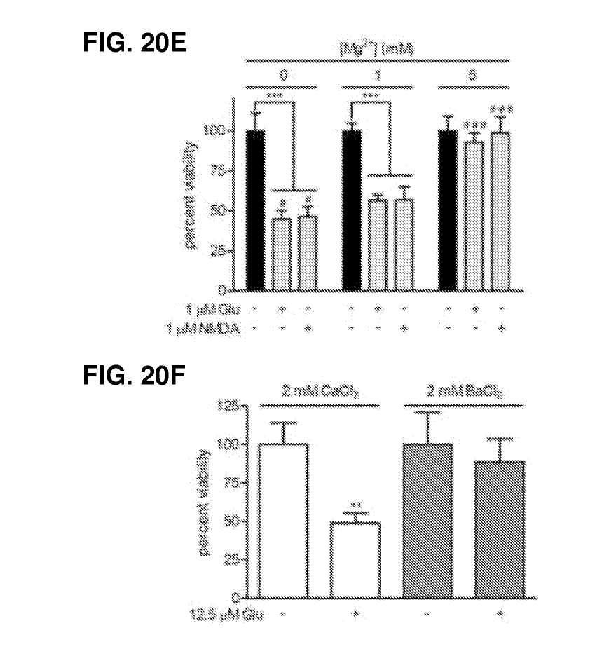

To determine whether glutamate treatment was neurotoxic to ESNs, metabolic activity was measured at 2, 6 and 24 h after the tonic application of 0.78-200 μM glutamate (FIG. 20A). At all doses metabolic failure was apparent by 2 h and complete by 24 h, with a calculated EC₅₀ of 0.41 μM (R²=0.99). To determine whether transient exposure would also cause toxicity, ESNs were exposed to 0.78-200 μM glutamate for 5 min, followed by incubation in glutamate-free media. As with the tonic treatment, metabolic inhibition was complete at 24 h for all doses above 0.78 μM glutamate, although the rate of cell death was delayed in the pulse treatment (FIG. 20B). Tonic addition of NMDA likewise resulted in a dose-dependent loss of ESN viability by 24 h, with a calculated EC₅₀ of 0.39 μM (R²=0.95).

Exposure of ESNs to subtype-specific agonists and antagonists was used to determine whether specific iGluRs were responsible for glutamate neurotoxicity. Whereas CNQX addition was not protective, APV treatment prevented glutamate-induced cell death in a dose-dependent manner (FIG. 20C). Notably, inhibition of VGCCs by addition of Gd³⁺ had no effect on viability (FIG. 20C). To verify the role of NMDARs in toxicity, we showed that NMDA-induced neurotoxicity was blocked in a dose-dependent manner by APV (FIG. 20D). Collectively, these data showed that glutamate-induced neurotoxicity was inhibited by iGluR antagonists in a dose-dependent manner, and further suggested that NMDAR activation was sufficient to cause neuron death.

To further evaluate the role of Ca²⁺ uptake by NMDARs in glutamate-induced neurotoxicity, cell viability assays were conducted by a 5 min treatment with 1 μM glutamate or NMDA in 0, 1 or 5 mM Mg²⁺, followed by wash-out and incubation in fresh NBA-B27 medium for 6 h. Cell death was potentiated in Mg²⁺-free medium, and significantly reduced in 5 mM Mg²⁺ (FIG. 20E). A causative role for Ca²⁺ in excitotoxicity was tested by substitution of Ba²⁺ for extracellular Ca²⁺, which conferred full protection against a 5 min treatment with 12.5 μM glutamate (FIG. 20F). These data demonstrate that induction of excitotoxicity is dependent on Ca²⁺ uptake, and show that methods to reduce the open state of the NMDAR channel concomitantly reduce Ca²⁺-mediated excitotoxicity.

### Example 16

### Glutamate Treatment Evokes Morphologic and Genetic Markers of Neurotoxicity

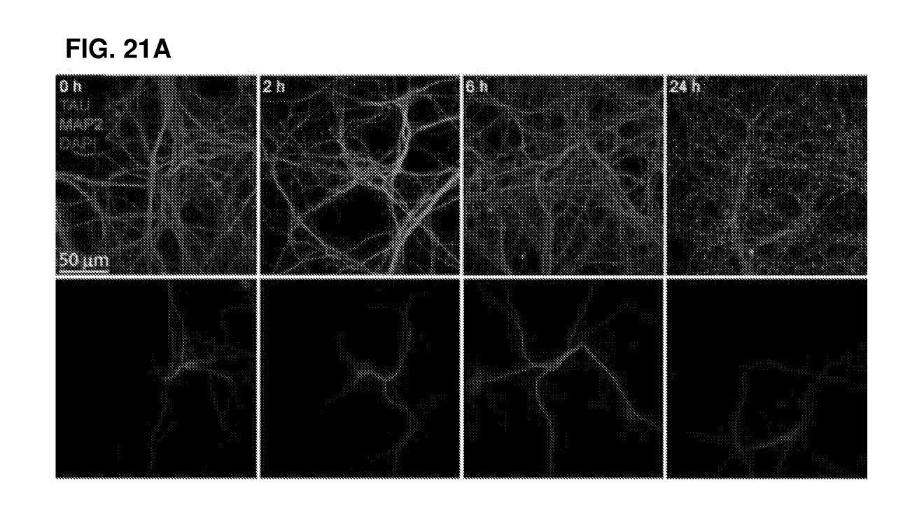

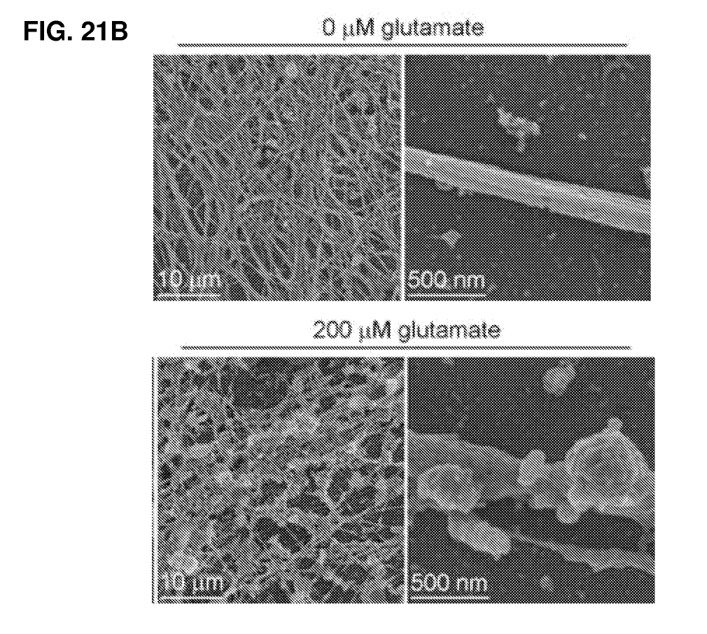

Glutamate-induced changes in neurite morphologies were characterized by immunofluorescent staining for the dendritic/somatic marker MAP2 and the axonal marker TAU protein. Although there was no apparent morphological evidence of neurotoxicity at 2 h, varicosity formation in axons increased 370% at 6 h (p<0.05) and 1,100% at 24 h (p<0.01), aid axonal degeneration was visually extensive by 24 h (FIG. 21A, upper panels). No gross changes in dendrite morphology were apparent, although MAP2-staining was less intense at 6 and 24 h (FIG. 21A, lower panels). Neurite degeneration was further evaluated using scanning electron microscopy (SEM) of neurons treated with 200 μM glutamate for 6 h (FIG. 21B). In contrast to the well-defined axodendritic processes in control neurons, glutamate evoked significant changes in morphology, including neurite fragmentation and loss somatic blebbing, varicosity formation, ruffled membranes and focal neurite swelling.

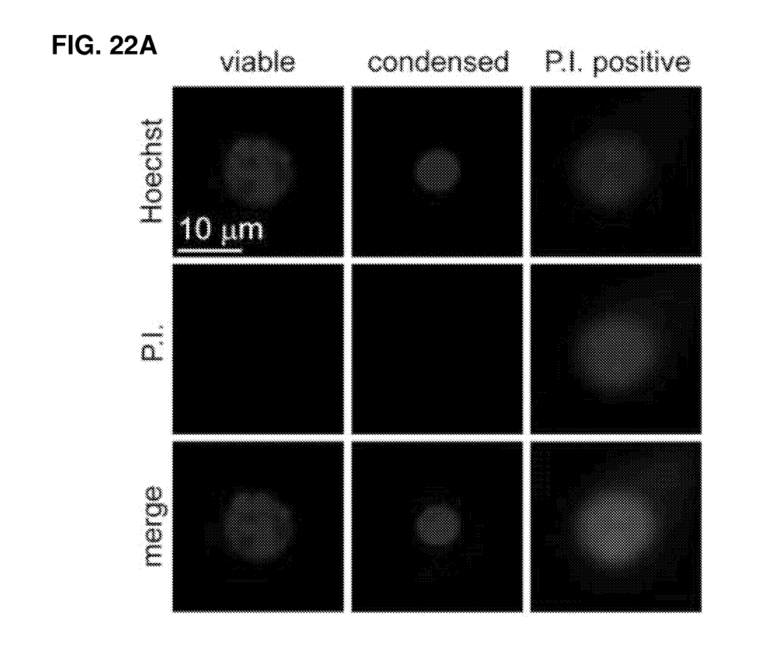

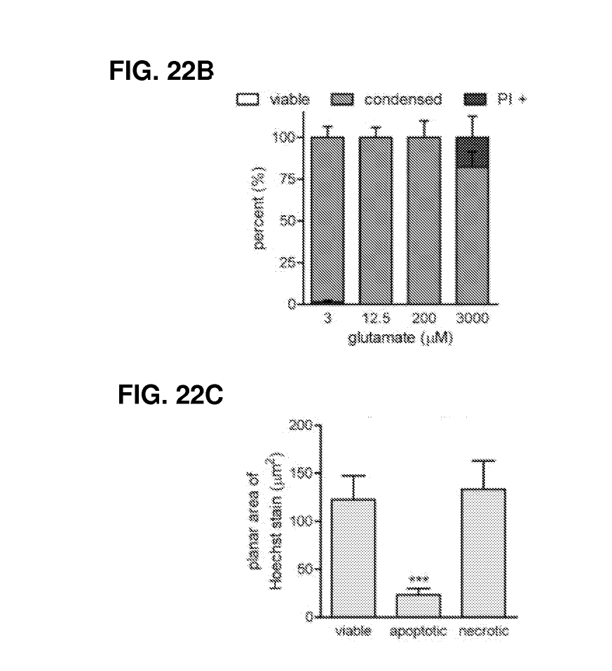

The paring of fluorescent membrane-permeant and impermeant nuclear dyes was used to evaluate the mode of excitogenic cell death (FIG. 22A). Tonic addition of 3-200 μM glutamate resulted in pyknotic, PI-negative nuclei at 2 h, indicative of apoptosis (FIG. 22B). Evidence of primary necrosis (non-pyknotic, PI-positive nuclei) was first observed with 3000 μM glutamate, which produced a mixture of necrotic and apoptotic neurons (FIG. 22B,C).

QPCR was used to confirm that transcripts associated with cell stress and death were up regulated at 6 h after tonic addition of 200 μM glutamate (Table 6), including caspases and genes associated with the regulation of apoptotic and autophagic cell death.

TABLE 6
QPCR analysis of neuronal and stress response genes.

| gene | Normalize log₂ (FC) | function |
|---|---|---|
| Grin1 | 0.39 | Synaptic activity |
| Snap25 | −0.52 | |
| Casp4 | 2.96 | Cell stress/death |
| Casp6 | 2.15 | |
| Casp12 | 3.03 | |
| Cdk1 | 3.11 | |
| CflaR | 2.54 | |
| Dap1 | 2.84 | |
| Dapk2 | 4.18 | |
| Dram1 | 2.63 | |
| Fadd1 | 1.98 | |
| Pawr | 3.49 | |
| Traf1 | 5.02 | |

### Example 17

### Inhibition of Glutamate-Induced Neurotoxicity

Protection against glutamate-induced neurotoxicity was evaluated in ESNs plated in 24-48 well dishes. Gd³⁺ was not used in these experiments because it did not appear to have an effect on neurotoxicity and to avoid masking an intrinsic neuroprotective effect induced by synaptic activity and/or possibly modulated by treatments.

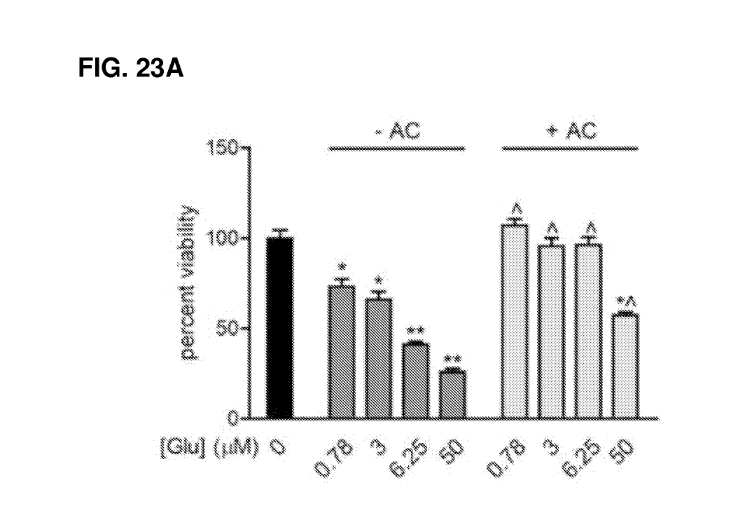

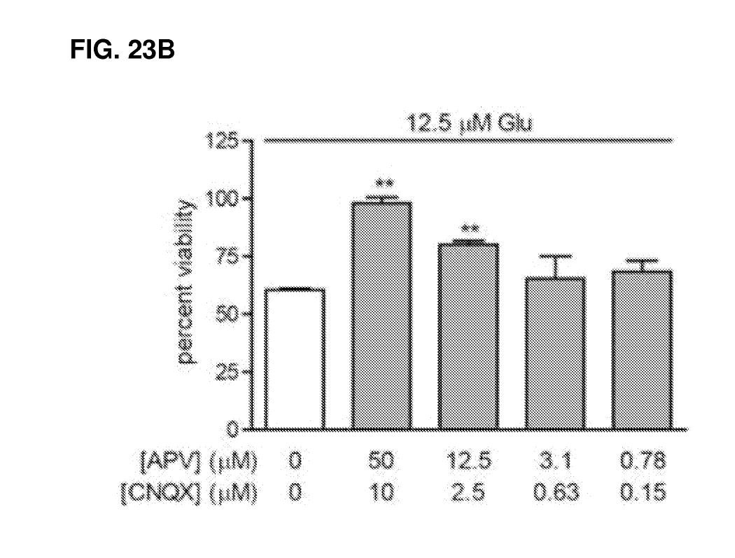

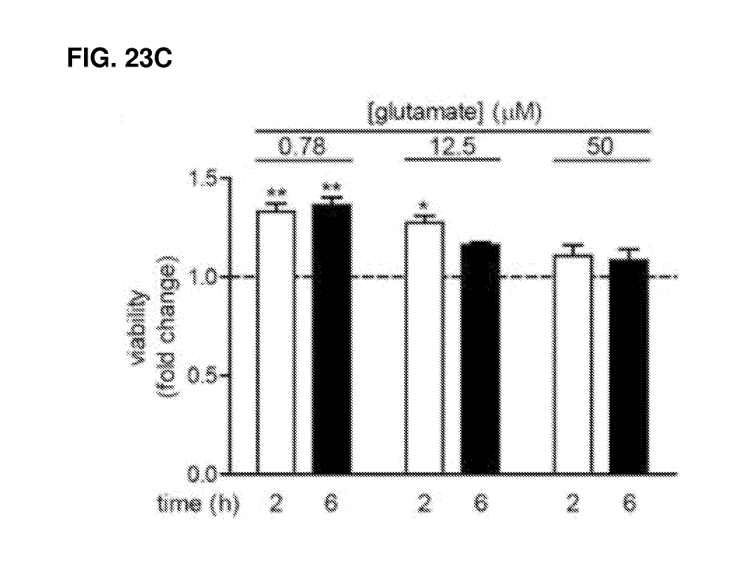

ESN viability was measured 6 h after administration of 50 μM APV+10 μM CNQX (1×A/C) and 0.78-50 μM glutamate. Full protection was conferred by 1×A/C against excitotoxicity up to 12.5 μM glutamate and resulted in reduced toxicity at 50 μM glutamate (FIG. 23A). In the reverse experiment, neurons were administered 12.5 μM glutamate with dilutions of 1×A/C, and neuron viability was measured at 2 h (FIG. 23B). Whereas 1×A/C conferred full protection against 12.5 μM glutamate, a four-fold decrease in A/C concentration reduced viability by 20%, and subsequent dilutions had no significant protective effect.

Next, the ability of a cocktail of neurotrophic factors (NTFs) to delay glutamate-mediated neurotoxicity was analyzed. Although the prophylactic efficacy of these NTFs is unknown with respect to excitogenic injury, activation of cognate neurotrophic receptors is neuroprotective in primary neuron cultures. NTFs were chosen based on three criteria: !) their efficacy had not previously been evaluated in excitotoxic injury; 2) several of the cognate receptors have been implicated in excitotoxic neuroprotection; and, 3) ESNs express transcripts for the cognate receptors. ESNs were pre-treated with NT3, BNDF, GNDF and CNTF for 16 h, and neuron viability was measured at 2, 6 and 24 h after addition of 0.78, 12.5 and 50 μM glutamate. NTF prophylaxis conferred partial protection against tonic glutamate treatment in a dose-dependent manner at 2 and 6 h (FIG. 23C). No significant protective effect was apparent at 24 h, indicating a limited efficacy during tonic dosing.

### Example 18

### Tetanus Toxin

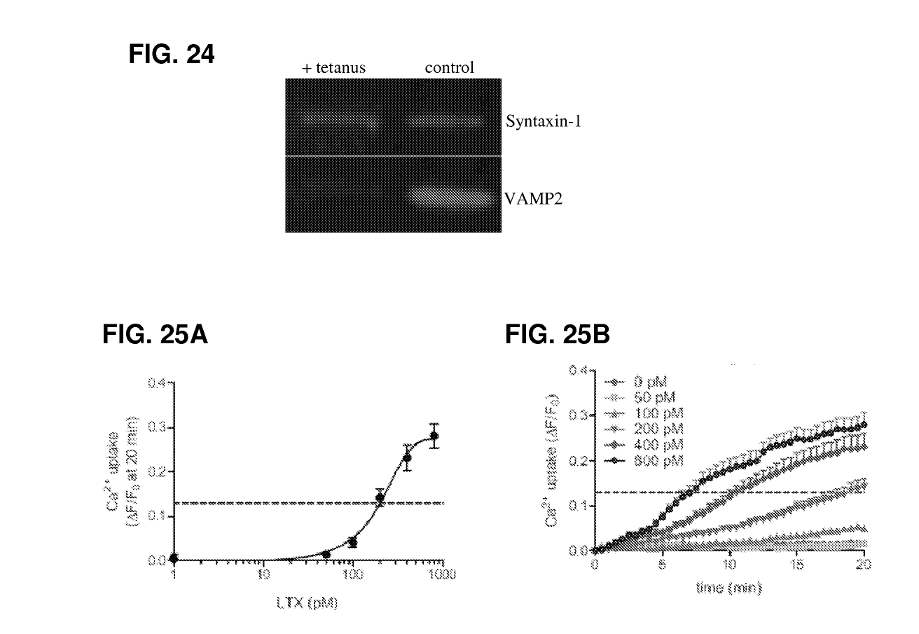

Tetanospasmin neurotoxin (tetanus) is a closely related cousin of botulinum neurotoxin. Similar to BoNTs, tetanus is internalized by binding SV2 and acts to specifically cleave VAMP2 (the same target as BoNT/B, /D, /F and /G). Based on immunoblot analysis, exposure to 150 pM tetanus for 24 h results in nearly complete loss of VAMP2 signal (FIG. 24). The loss of signal is due to the rapid proteolytic clearance of the cleaved fragment of VAMP-2.

### Example 19

### Screening for Small Molecular Therapeutics that Reduce Neuronal Response to Black Widow Spider Venom

The black widow species (e.g., L. mactans or L. hesperus) are particularly dangerous due to the large quantities of toxin delivered per bite, and are considered the most venomous spiders in the world. Their venom is 15 times more concentrated than that of the prairie rattlesnake and ranks among the most potent toxins secreted by an animal. The most active component of black widow spider venom is the neurotropic neurotoxin α-latrotoxin (LTX), which acts as a highly potent secretagogue that induces fulminant neurotransmitter release at central and autonomic synapses. There is a dose-response relationship between LTX, Ca2+ influx and neurotoxicity in ESNs (FIG. 25).

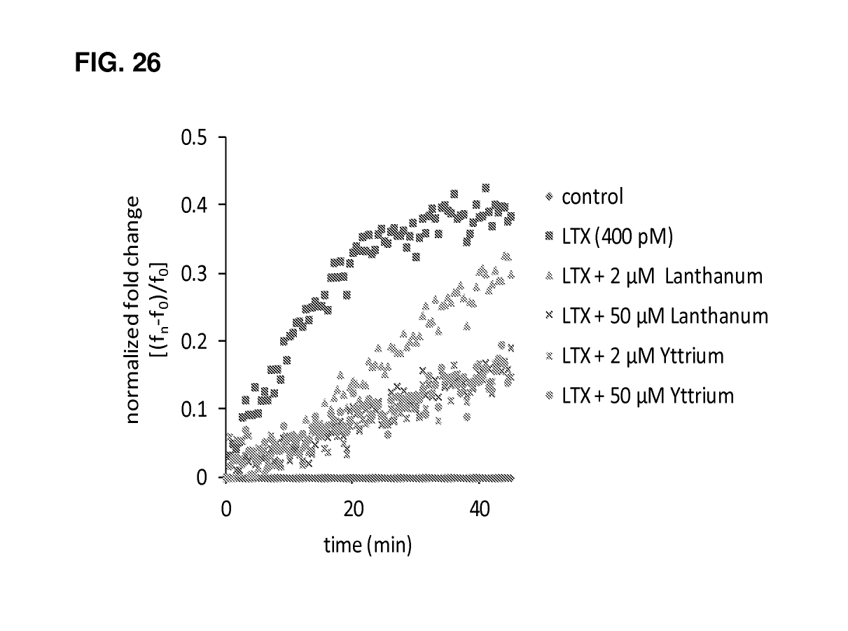

Furthermore, a method to evaluate the ability of candidate therapeutics to inhibit Ca²⁺ influx through LTX pores has been developed (FIG. 26 and Table 7). In a sample screening, it was determined that Y³⁺ was most effective at reducing Ca²⁺ influx at less than 2 μM, which is more than 2.000-fold lower than the reported intraperitoneal LD₅₀ for Y³⁺ in rats, suggesting that Y³⁺ model may be a therapeutic candidate for mitigating the morbidity associated with black widow spider bites.

TABLE 7
Summary of the effect of ten ions on Ca²⁺
internalization into neurons following LTX treatment.

| Metal ion | [symbol] influx, normalized to LTX (%) |
|---|---|
| LTX | 100.0 |
| 50 μM [symbol] | 47.5 |
| 50 μM Sc³⁺ | 71.3 |
| 50 μM Tb³⁺ | 66.1 |
| 50 μM [symbol] | 40.9 |
| 50 μM [symbol] | 48.7 |
| 50 μM Ba²⁺ | 94.5 |
| 50 μM Cs¹⁺ | 111.5 |
| 50 μM Ni²⁺ | 97.5 |
| 50 μM Nd³⁺ | 174.8 |
| 50 μM [symbol] | 48.6 |
| 2 μM [symbol] | 42.5 |

Those ions that reduced Ca²⁺ influx by more than 50% are in bold. Note that co-administration of Nd³⁺ significantly potentiated Ca²⁺ influx.

### Example 20

### Identify/Characterize Neurotoxins that Inhibit/Potential Propagation of the Action Potential

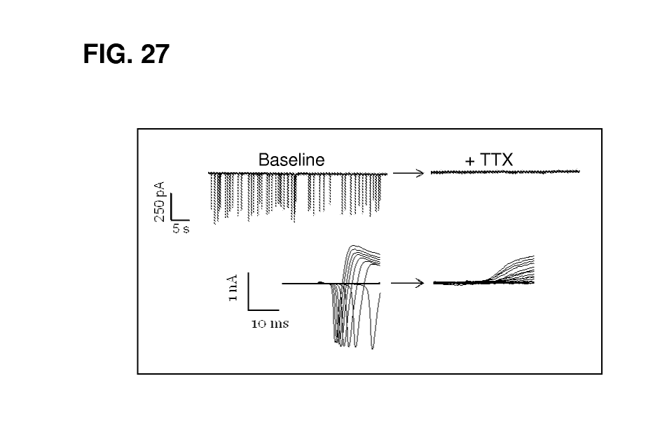

Tetrodotoxin (TTX) blocks action potentials in nerves by binding to the voltage-gated, fast sodium channels that propagate the action potential, essentially preventing intoxicated neurons from firing. TTX can be identified electrophysiologically by the inhibition of Na⁺-gated currents under depolarizing conditions in the presence of TTX (FIG. 27).

### Example 21

### Identify/Characterize Drugs that Modulate GABA Receptor Activity

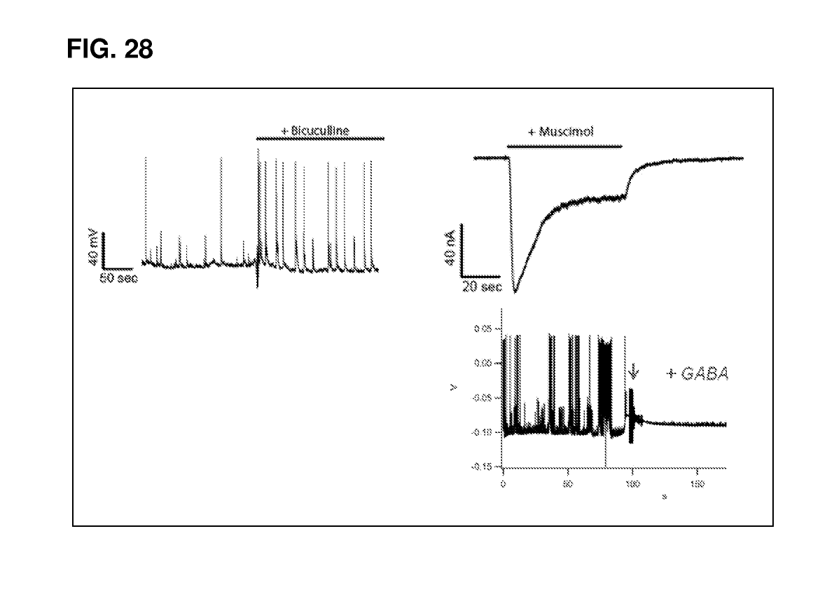

Along with glutamatergic neurons, the GABAergic neuron family is critical for normal central nervous system activity. Using RNAseq we have shown that ESNs express the necessary subunits to form functional GABA-A and GABA-B receptors, though not GABA-C (Table 8 and FIG. 28). Notably, ESNs exhibit a GABA-A current in the presence of the active component of psilocybin mushrooms, muscimol, and inhibition of GABA activity in the presence of bicuculline.

TABLE 8
ESNs express necessary elements to form functional GABA receptors.

| Subunits | | DIV −8 | DIV −4 | DIV 0 | DIV 1 | DIV 7 | DIV 16 | DIV 18 | DIV 21 | DIV 28 |
|---|---|---|---|---|---|---|---|---|---|---|
| GABA-A | Gabra1 | 4 | 4 | 24 | 52 | 206 | 1729 | 1506 | 2941 | 2835 |
| receptor | Gabra2 | 5 | 2 | 26 | 77 | 1228 | 1362 | 1243 | 1263 | 1157 |
| | Gabra3 | 14 | 18 | 80 | 89 | 1427 | 2415 | 2285 | 2287 | 2588 |
| | Gabra4 | 62 | 25 | 49 | 22 | 786 | 685 | 867 | 619 | 549 |
| | Gabra5 | 30 | 7 | 30 | 40 | 3233 | 3025 | 3670 | 2729 | 2062 |
| | Gabrb1 | 1 | 0 | 1 | 2 | 83 | 157 | 145 | 152 | 96 |
| | Gabrb2 | 8 | 11 | 254 | 402 | 2121 | 5794 | 4676 | 8478 | 7241 |
| | Gabrb3 | 216 | 182 | 421 | 1523 | 7482 | 7845 | 8024 | 6889 | 5736 |
| | Gabrd | 5 | 7 | 1 | 1 | 2 | 2 | 2 | 3 | 3 |
| | Gabre | 13 | 13 | 13 | 9 | 14 | 41 | 40 | 36 | 69 |
| | Gabrg1 | 9 | 4 | 59 | 14 | 140 | 243 | 225 | 197 | 181 |
| | Gabrg2 | 5 | 1 | 74 | 376 | 220 | 1270 | 687 | 1259 | 1136 |
| | Gabrg3 | 3 | 3 | 1 | 2 | 85 | 231 | 235 | 205 | 135 |
| | Gabrp | 27 | 16 | 2 | 0 | 1 | 1 | 0 | 0 | 0 |
| | Gabrq | 2 | 2 | 9 | 7 | 44 | 143 | 176 | 106 | 105 |
| GABA-B | Gabbr1 | 309 | 596 | 1470 | 4130 | 13328 | 19471 | 19586 | 18726 | 14777 |
| receptor | Gabbr2 | 2 | 3 | 40 | 78 | 2026 | 3791 | 3714 | 3358 | 2561 |
| GABA-C | Gabrr1 | 25 | 4 | 2 | 1 | 1 | 2 | 2 | 8 | 11 |
| receptor | Gabrr2 | 2 | 0 | 3 | 4 | 5 | 3 | 4 | 3 | 6 |
| | Gabrr3 | 0 | 1 | 0 | 0 | 0 | 1 | 0 | 0 | 0 |

The invention illustratively disclosed herein suitably may be practiced in the absence of any element, which is not specifically disclosed herein. It is apparent to those skilled in the art, however, that many changes, variations, modifications, other uses, and applications to the method are possible, and also changes, variations, modifications, other uses, and applications which do not depart from the spirit and scope of the invention are deemed to be covered by the invention, which is limited only by the claims which follow.

1. A method of detecting toxin activity comprising:
a. contacting a sample to a neuron population, wherein the neuron population is capable of synaptic activity, and wherein the neuron population is derived from a population of pluripotent cells; and,
b. detecting the presence of toxin activity in the contacted neuron population relative to a control neuron population, wherein a difference in the toxin activity of the contacted neuron population as compared to the control neuron population is indicative of toxin in the sample.

2. The method of claim 1, wherein the neuron population comprises neurons susceptible to toxin intoxication by about 100 pM or less of a toxin.

3. The method of claim 1, wherein the neuron population comprises neurons susceptible to toxin intoxication by about 10 pM or less of a toxin.

4. The method of claim 1, wherein the neuron population comprises neurons susceptible to toxin intoxication by about 0.1 pM or less of a toxin.

5. (canceled)

6. The method of claim 1, wherein the toxin is selected from the group consisting of botulinum toxin, tetanus toxin, latrotoxin, shiga toxin, tetrodotoxin, conotoxin, and combinations thereof.

7. The method of claim 6, wherein the botulinum toxin is selected from the group consisting of serotype /A, serotype /B, serotype /C, serotype /D, serotype /E, serotype /F, serotype /G, or combinations thereof.

8. The method of claim 1, wherein the sample is selected from the group consisting of a purified toxin, a partially purified toxin, or unpurified toxin.

9. The method of claim 1, wherein the sample is selected from the group consisting of a bulk toxin, a formulated toxin, a cosmetics toxin formulation, or a clinical toxin formulation.

10. The method of claim 1, wherein the sample is selected from the group consisting of a raw food, a cooked food, a partially cooked food, a processed food, or combinations thereof.

11. The method of claim 1, wherein the sample is taken from a subject.

12. The method of claim 4, wherein the sample is selected from the group consisting tissue, saliva, excretion, feces, blood, urine, or combinations thereof.

13. A method of screening for a molecule able to alter toxin activity comprising:
a. contacting a neuron population with toxin forming an exposed neuron population, wherein the neuron population is derived from a population of pluripotent cells;
b. detecting the activity of the toxin of the exposed neuron population;
c. contacting the exposed neuron population with a test molecule forming a test neuron population;
d. measuring the toxin activity of the test neuron population;
e. comparing the toxin activity of the test neuron population to the toxin activity of the exposed neuron population; and,
f. correlating a decrease in activity to a molecule capable of inhibiting the toxin activity, or correlating an increase in activity to a molecule capable of enhancing toxin activity.

14. (canceled)

15. (canceled)

16. (canceled)

17. (canceled)

18. (canceled)

19. (canceled)

20. (canceled)

21. A method of detecting neurotoxicity comprising:
a. contacting a sample to a neuron population, wherein the neuron population is capable of synaptic activity, and wherein the neuron population is derived from a population of pluripotent cells; and,
b. detecting the metabolic activity of the contacted neuron population relative to a control neuron population, wherein a difference in the metabolic activity of the contacted neuron population as compared to the control neuron population is indicative of neurotoxicity.

22. (canceled)

23. (canceled)

24. (canceled)

25. (canceled)

26. (canceled)

27. (canceled)

28. (canceled)

29. (canceled)

30. (canceled)

31. The method of claim 21, wherein the sample is taken from an environment.

32. The method of claim 21, wherein the environment sample is selected from the group consisting of soil, water, biomass, plant, tree, air, gas, or combinations thereof.

33. A composition comprising an isolated population of neurons derived in vitro from at least one pluripotent cell, wherein in the pluripotent cell is selected from the group consisting of non-murine embryonic stem cell, induced pluripotent stem cell, adult stem cell and combinations thereof.

34. The composition of claim 33, wherein the neurons form synapses.

35. The composition of claim 33, wherein the neurons release neurotransmitters.

36. The composition of claim 33, wherein the neurons produce cell to cell signals.

37. A kit comprising:
a. an isolated population of neurons derived in vitro from at least one pluripotent cell; and,
b. a container.

38. The kit of claim 37 further comprising culture media.

39. The kit of claim 37 further comprising a positive control toxin.
